SEQUENCE LISTING (23 sequences)

SEQ ID NO: 1 - protein, 1080 aa, Artificial Sequence
  fusion two protein
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHALHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEGSGGVDDEEELIQLIIEKTGKSREEIEKMVEEKIKAFNNLISRRGALLLVAKKLGVLYKNTPKEKKIGELESWEYVKVKGKILKSFGLISYSKGKFQPIILGDETGTIKAIIWNTDKELPENTVIEAIGKTKINKKTGNLELHIDSYKILESDLEIKPQKQEFVGICIVKYPKKQTQKGTIVSKAILTSLDRELPVVYFNDFDWEIGHIYKVYGKLKKNIKTGKIEFFADKVEEATLKDLKAFKGEAD

SEQ ID NO: 2 - protein, 1021 aa, Artificial Sequence
  fusion two protein
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHALHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEGSGGVDKIGELESWEYVKVKGKILKSFGLISYSKGKFQPIILGDETGTIKAIIWNTDKELPENTVIEAIGKTKINKKTGNLELHIDSYKILESDLEIKPQKQEFVGICIVKYPKKQTQKGTIVSKAILTSLDRELPVVYFNDFDWEIGHIYKVYGKLKKNIKTGKIEFFADKVEEATLKDLKAFKGEAD

SEQ ID NO: 3 - protein, 930 aa, Artificial Sequence
  fusion two protein
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHALHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEGSGGVDKPQKQEFVGICIVKYPKKQTQKGTIVSKAILTSLDRELPVVYFNDFDWEIGHIYKVYGKLKKNIKTGKIEFFADKVEEATLKDLKAFKGEAD

SEQ ID NO: 4 - protein, 6 aa, Artificial Sequence
  Linker
GSGGVD

SEQ ID NO: 5 - protein, 832 aa, Thermus aquaticus
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHALHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 6 - protein, 243 aa, Nanoarchaeum equitans
MDEEELIQLIIEKTGKSREEIEKMVEEKIKAFNNLISRRGALLLVAKKLGVLYKNTPKEKKIGELESWEYVKVKGKILKSFGLISYSKGKFQPIILGDETGTIKAIIWNTDKELPENTVIEAIGKTKINKKTGNLELHIDSYKILESDLEIKPQKQEFVGICIVKYPKKQTQKGTIVSKAILTSLDRELPVVYFNDFDWEIGHIYKVYGKLKKNIKTGKIEFFADKVEEATLKDLKAFKGEAD

SEQ ID NO: 7 - protein, 92 aa, Nanoarchaeum equitans
MKIGELESWEYVKVKGKILKSFGLISYSKGKFQPIILGDETGTIKAIIWNTDKELPENTVIEAIGKTKINKKTGNLELHIDSYKILESDLEI

SEQ ID NO: 8 - protein, 93 aa, Nanoarchaeum equitans
MKPQKQEFVGICIVKYPKKQTQKGTIVSKAILTSLDRELPVVYFNDFDWEIGHIYKVYGKLKKNIKTGKIEFFADKVEEATLKDLKAFKGEAD

SEQ ID NO: 9 - DNA, 8539 bp, Artificial Sequence
  Fusion of two diffrent gene
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatatacatatgagggggatgctgcccctctttgagcccaagggccgggtcctcctggtggacggccaccacctggcctaccgcaccttccacgccctgaagggcctcaccaccagccggggggagccggtgcaggcggtctacggcttcgccaagagcctcctcaaggccctcaaggaggacggggacgcggtgatcgtggtctttgacgccaaggccccctccttccgccacgaggcctacggggggtacaaggcgggccgggcccccacgccggaggactttccccggcaactcgccctcatcaaggagctggtggacctcctggggctggcgcgcctcgaggtcccgggctacgaggcggacgacgtcctggccagcctggccaagaaggcggaaaaggagggctacgaggtccgcatcctcaccgccgacaaagacctttaccagctcctttccgaccgcatccacgccctccaccccgaggggtacctcatcaccccggcctggctttgggaaaagtacggcctgaggcccgaccagtgggccgactaccgggccctgaccggggacgagtccgacaaccttcccggggtcaagggcatcggggagaagacggcgaggaagcttctggaggagtgggggagcctggaagccctcctcaagaacctggaccggctgaagcccgccatccgggagaagatcctggcccacatggacgatctgaagctctcctgggacctggccaaggtgcgcaccgacctgcccctggaggtggacttcgccaaaaggcgggagcccgaccgggagaggcttagggcctttctggagaggcttgagtttggcagcctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacggagaagaccggcaagcgctccaccagcgccgccgtcctggaggccctccgcgaggcccaccccatcgtggagaagatcctgcagtaccgggagctcaccaagctgaagagcacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggagaccgccagctggatgttcggcgtcccccgggaggccgtggaccccctgatgcgccgggcggccaagaccatcaacttcggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggagaccctcttcggccgccgccgctacgtgccagacctagaggcccgggtgaagagcgtgcgggaggcggccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggagggcagcggtggcgttgatgatgaagaggaactaatacaactaataatagaaaaaactggcaaatctcgagaggaaatagaaaaaatggtggaagaaaaaattaaagcttttaacaatttaatatctcgtaggggggctttactattagtagcaaaaaaacttggtgttttgtataaaaacactccgaaagagaaaaaaattggcgaattagaaagctgggaatatgtaaaagtaaagggcaaaattctcaaatcttttggattaattagttattcgaaagggaaattccaacctattattttaggagacgaaaccggtactattaaagctattatttggaataccgataaagaattacctgaaaacactgtaatagaagctattgggaaaaccaaaattaataagaaaactggcaatttagaattacatatagacagttataaaattttagaaagcgatttagagataaaaccccaaaagcaagaatttgttgggatttgcatagttaaatatccaaaaaaacaaacccaaaaaggcacaatagtatcgaaagcaattttaactagcttagatagggaattgcctgtagtatatttcaacgattttgattgggaaataggccatatatataaagtatatggaaagcttaagaaaaacataaaaactggtaaaatagaatttttcgctgacaaagttgaggaagcaacattaaaagatctaaaagcttttaaaggagaggccgatcaccaccaccaccaccactaaggatccgaattcgagctccgtcgacaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 10 - DNA, 8362 bp, Artificial Sequence
  Fusion of two diffrent gene
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatatacatatgagggggatgctgcccctctttgagcccaagggccgggtcctcctggtggacggccaccacctggcctaccgcaccttccacgccctgaagggcctcaccaccagccggggggagccggtgcaggcggtctacggcttcgccaagagcctcctcaaggccctcaaggaggacggggacgcggtgatcgtggtctttgacgccaaggccccctccttccgccacgaggcctacggggggtacaaggcgggccgggcccccacgccggaggactttccccggcaactcgccctcatcaaggagctggtggacctcctggggctggcgcgcctcgaggtcccgggctacgaggcggacgacgtcctggccagcctggccaagaaggcggaaaaggagggctacgaggtccgcatcctcaccgccgacaaagacctttaccagctcctttccgaccgcatccacgccctccaccccgaggggtacctcatcaccccggcctggctttgggaaaagtacggcctgaggcccgaccagtgggccgactaccgggccctgaccggggacgagtccgacaaccttcccggggtcaagggcatcggggagaagacggcgaggaagcttctggaggagtgggggagcctggaagccctcctcaagaacctggaccggctgaagcccgccatccgggagaagatcctggcccacatggacgatctgaagctctcctgggacctggccaaggtgcgcaccgacctgcccctggaggtggacttcgccaaaaggcgggagcccgaccgggagaggcttagggcctttctggagaggcttgagtttggcagcctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacggagaagaccggcaagcgctccaccagcgccgccgtcctggaggccctccgcgaggcccaccccatcgtggagaagatcctgcagtaccgggagctcaccaagctgaagagcacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggagaccgccagctggatgttcggcgtcccccgggaggccgtggaccccctgatgcgccgggcggccaagaccatcaacttcggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggagaccctcttcggccgccgccgctacgtgccagacctagaggcccgggtgaagagcgtgcgggaggcggccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggagggcagcggtggcgttgataaaattggcgaattagaaagctgggaatatgtaaaagtaaagggcaaaattctcaaatcttttggattaattagttattcgaaagggaaattccaacctattattttaggagacgaaaccggtactattaaagctattatttggaataccgataaagaattacctgaaaacactgtaatagaagctattgggaaaaccaaaattaataagaaaactggcaatttagaattacatatagacagttataaaattttagaaagcgatttagagataaaaccccaaaagcaagaatttgttgggatttgcatagttaaatatccaaaaaaacaaacccaaaaaggcacaatagtatcgaaagcaattttaactagcttagatagggaattgcctgtagtatatttcaacgattttgattgggaaataggccatatatataaagtatatggaaagcttaagaaaaacataaaaactggtaaaatagaatttttcgctgacaaagttgaggaagcaacattaaaagatctaaaagcttttaaaggagaggccgatcaccaccaccaccaccactaaggatccgaattcgagctccgtcgacaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 11 - DNA, 8089 bp, Artificial Sequence
  Fusion of two diffrent gene
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatatacatatgagggggatgctgcccctctttgagcccaagggccgggtcctcctggtggacggccaccacctggcctaccgcaccttccacgccctgaagggcctcaccaccagccggggggagccggtgcaggcggtctacggcttcgccaagagcctcctcaaggccctcaaggaggacggggacgcggtgatcgtggtctttgacgccaaggccccctccttccgccacgaggcctacggggggtacaaggcgggccgggcccccacgccggaggactttccccggcaactcgccctcatcaaggagctggtggacctcctggggctggcgcgcctcgaggtcccgggctacgaggcggacgacgtcctggccagcctggccaagaaggcggaaaaggagggctacgaggtccgcatcctcaccgccgacaaagacctttaccagctcctttccgaccgcatccacgccctccaccccgaggggtacctcatcaccccggcctggctttgggaaaagtacggcctgaggcccgaccagtgggccgactaccgggccctgaccggggacgagtccgacaaccttcccggggtcaagggcatcggggagaagacggcgaggaagcttctggaggagtgggggagcctggaagccctcctcaagaacctggaccggctgaagcccgccatccgggagaagatcctggcccacatggacgatctgaagctctcctgggacctggccaaggtgcgcaccgacctgcccctggaggtggacttcgccaaaaggcgggagcccgaccgggagaggcttagggcctttctggagaggcttgagtttggcagcctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacggagaagaccggcaagcgctccaccagcgccgccgtcctggaggccctccgcgaggcccaccccatcgtggagaagatcctgcagtaccgggagctcaccaagctgaagagcacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggagaccgccagctggatgttcggcgtcccccgggaggccgtggaccccctgatgcgccgggcggccaagaccatcaacttcggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggagaccctcttcggccgccgccgctacgtgccagacctagaggcccgggtgaagagcgtgcgggaggcggccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggagggcagcggtggcgttgataaaccccaaaagcaagaatttgttgggatttgcatagttaaatatccaaaaaaacaaacccaaaaaggcacaatagtatcgaaagcaattttaactagcttagatagggaattgcctgtagtatatttcaacgattttgattgggaaataggccatatatataaagtatatggaaagcttaagaaaaacataaaaactggtaaaatagaatttttcgctgacaaagttgaggaagcaacattaaaagatctaaaagcttttaaaggagaggccgatcaccaccaccaccaccactaaggatccgaattcgagctccgtcgacaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 12 - DNA, 48 bp, Artificial Sequence
  TaqPolNeqSSBFull_F1
aactttaagaaggagatatacatatgagggggatgctgcccctctttg

SEQ ID NO: 13 - DNA, 41 bp, Artificial Sequence
  TaqPolNeqSSBFull_R1
tcatcaacgccaccgctgccctccttggcggagagccagtc

SEQ ID NO: 14 - DNA, 51 bp, Artificial Sequence
  TaqPolNeqSSBFull_F2
agggcagcggtggcgttgatgatgaagaggaactaatacaactaataatag

SEQ ID NO: 15 - DNA, 80 bp, Artificial Sequence
  TaqPolNeqSSBFull_R2
gcaagcttgtcgacggagctcgaattcggatccttagtggtggtggtggtggtgatcggcctctcctttaaaagctttta

SEQ ID NO: 16 - DNA, 48 bp, Artificial Sequence; identical to SEQ ID NO: 12
  TaqPolNeqSSBII+III_F1

SEQ ID NO: 17 - DNA, 41 bp, Artificial Sequence; identical to SEQ ID NO: 13
  TaqPolNeqSSBII+III_R1

SEQ ID NO: 18 - DNA, 51 bp, Artificial Sequence; identical to SEQ ID NO: 14
  TaqPolNeqSSBII+III_F2

SEQ ID NO: 19 - DNA, 80 bp, Artificial Sequence; identical to SEQ ID NO: 15
  TaqPolNeqSSBII+III_R2

SEQ ID NO: 20 - DNA, 48 bp, Artificial Sequence; identical to SEQ ID NO: 12
  TaqPolNeqSSBIII_F1

SEQ ID NO: 21 - DNA, 41 bp, Artificial Sequence
  TaqPolNeqSSBIII_R1
ttatcaacgccaccgctgccctccttggcggagagccagtc

SEQ ID NO: 22 - DNA, 45 bp, Artificial Sequence
  TaqPolNeqSSBIII_F2
agggcagcggtggcgttgataaaccccaaaagcaagaatttgttg

SEQ ID NO: 23 - DNA, 70 bp, Artificial Sequence
  TaqPolNeqSSBIII_R2
gcaagcttgtcgacggagctcgaattcggatccttagtggtggtggtggtggtgatcggcctctccttta

(19) United States
(12) Patent Application Publication
(10) Pub. No.: US 2024/0240163 A1
(43) Pub. Date: Jul. 18, 2024

(54) TAQ-NEQSSB POLYMERASE, THE METHOD OF ITS OBTAINING, RECOMBINANT PLASMID, PRIMERS, AND APPLICATION OF THE POLYMERASE

(71) Applicant: INSTYTUT BIOTECHNOLOGII l MEDYCYNY MOLEKULARNEJ, Gdansk (PL)

(72) Inventors: Marta SKWARECKA, Gdansk (PL); Grzegorz ZIELINSKI, Gdansk (PL); Kasjan SZEMIAKO, Gdansk (PL); Dawid NIDZWORSKI, Gdansk (PL); Sabina ZOLEDOWSKA, Gdansk (PL)

(21) Appl. No.: 18/562,014

(22) PCT Filed: May 18, 2022
(86) PCT No.: PCT/PL2022/000031

§ 371 Date: Nov. 17, 2023

(30) Foreign Application Priority Data
May 19, 2021 (PL) P.437909

(51) Int. Cl. C12N 9/12; C12N 15/62

(52) U.S. Cl. CPC C12N 9/1252; C12N 15/62; C07K 2319/80; C07K 2319/85; C12Y 207/07007

(57) ABSTRACT

The subject of the invention is a TaqPol-NeqSSB polymerase and its cloning method. Furthermore, the subject of the invention is an isolated recombinant plasmid, primers, and the application of the polymerase to replicate specific sequences of the SARS CoV-2 virus.

The subject of the invention is a TaqPol-NeqSSB polymerase and its cloning method. Furthermore, the subject of the invention is an isolated recombinant plasmid, primers, and the application of the polymerase to replicate specific sequences of the SARS CoV-2 virus.

SSB proteins (Single Stranded DNA Binding proteins) are present in all living organisms. They are involved in all processes that generate single-stranded DNA fragments, such as replication, recombination, and DNA repair. They protect single-stranded DNA (ssDNA) from degradation and simultaneously interact with other proteins in the cell. SSB-like proteins are known, which are proteins synthesised by mammalian, yeast, archaea, and bacterial cells. These proteins differ in molecular mass, number of subunits contained in the native molecule, size of the binding site, depending on their source of origin.

The mechanism of binding protein to ssDNA is based on the stacking interaction between aromatic amino acid residues and bases in the oligonucleotide chain, and the interaction of positively charged amino acid residues with the phosphate backbone in the ssDNA molecule. This bond is strong enough not to break down under the influence of low concentrations of NaCl. A high concentration of salt containing Mg2+ is required to break down the ssDNA-SSB complex in the cell. The length of the ssDNA fragment bound by SSB is shortened by 35%.

Although the NeqSSB protein belongs to the SSB family of proteins, it differs in its characteristics from classical SSB proteins and is therefore referred to as a NeqSSB-like protein. This protein originates from the hyperthermophilic archaeon Nanoarchaeum equitans, which parasitises on the craenarchaeon Ignicoccus hospitalis. Optimal growth conditions for this microorganism require strictly anaerobic conditions and a temperature of 90° C. Interestingly, Nanoarchaeum equitans has the smallest known genome consisting of 490,885 base pairs. Unlike most known organisms with reduced genomes, it possesses a full set of enzymes involved in DNA replication, repair and recombination, including the SSB protein.

The NeqSSB protein, like other proteins of this family, has the natural ability to bind DNA. It consists of 243 amino acid residues and contains one OB domain in its structure (FIG. 1). It shows biological activity as a monomer, similarly as in the case of some viral SSB proteins. The research shows that NeqSSB protein exhibits unusual capabilities concerning binding of all DNA forms (ssDNA, dsDNA), and mRNA without structural preferences. NeqSSB protein domains I, II, and III are responsible for binding to nucleotide acids—domains II, and III are responsible for the strongest binding domains. In addition, the protein is characterised by high thermostability. The half-life while maintaining the biological activity is 5 min in 100° C., while the melting temperature is 100.2° C.

DNA polymerase is an enzyme that plays an essential role in DNA replication and repair. It is used in the polymerase chain reaction (PCR), where it catalyses DNA synthesis process in vitro, being responsible for attaching subsequent nucleotides to the 3′OH end of the DNA strand. Besides the basic polymerisation capabilities, it can also show the ability to hydrolyse DNA molecules thanks to the presence of the exonucieolytic domain. Although polymerases perform the same functions, i.e., are responsible for DNA strand synthesis, their structures and activities differ significantly. They catalyse the mechanism of attaching subsequent nucleotides to the 3′OH end of the DNA strand. The structure of these polymerases, in bacteria, archaea, and eukaryotes, shows similarity regarding their structure and the presence of 3 basic subdomains of the finger, palm, and thumb, to which strictly defined functions are assigned. DNA polymerases vary in terms of the rate of catalysis, processivity, presence or absence of interacting protein subunits, or display of nucleolytic activity. The general division of DNA polymerases classifies them into 7 different families: A, B, C, D, X, Y, and RT. Members of bacterial DNA polymerases can be found in families A, B, C, X, Y, archaea in B, D, X, and Y, and eukaryotic DNA polymerases belong to families A, B, X, Y, and RT.

The primary task of DNA polymerase is to add nucleotides that are complementary to the 3′OH end of the DNA chain. The DNA polymerase mechanism of action includes several significant steps. The first one consists of the attachment of the enzyme to the DNA template. Obtained DNA-DNA complex associates the respective dNTPs (deoxyribonucleotide triphosphates) as the result of the nucleophilic attack of the 3′ OH end on the nucleotide phosphorus atom. The last step leads to the generation of the phosphodiester bond and the release of the pyrophosphate. The first step, that is the binding of the polymerase to the matrix, where the primer forces the thumb subdomain to change its conformation and fit tightly to the DNA molecule.

The thumb subdomain rotates with respect to the palm subdomain, and the conserved residues at the top of the thumb perform an opposite twist. In this way, the DNA polymerase interacts with a minor groove of DNA via the appropriately bent thumb subdomain. All of this causes the 3 bases within the primer to bend, and the DNA molecule to be close enough to the enzyme active site. The enhancement of the DNA strand bending is enabled by further interactions with the polymerase. This time, the palm subdomain determines the rotation of the first two bases of the DNA matrix by 900 and 1800 respectively, thus rotating the bases to the outside of the helix and forming an S-shaped conformation. Therefore, this conformation is induced by the interaction of the DNA with the thumb, and palm subdomain and the interaction of the matrix with the active centre.

The most used DNA polymerase of bacterial origin is Taq polymerase isolated from Thermus aquaticus. This enzyme, discovered in 1976, which revolutionised molecular biology, is made up of 832 amino acid residues with a molecular mass of 94 kDa. It is worth noting that its highest activity is achieved at temperatures between 72° C. and 80° C. Polymerisation is possible through the attachment of DNA to the active centre of the enzyme, in which the most relevant amino acid residues are Arg682, Lys785, Tyr766, Arg821, His811.

The Taq DNA polymerase has three domains: an exonucleolytic 5′→3′, an inactive exonucleolytic 3′→5′ and a polymerisation domain 5′→3′. The deletion of the exonucieolytic domain i.e., in Taq DNA polymerase allows for the obtaining of a functional protein with partially altered characteristics compared with the native enzyme. Without the 5′-3′ exonuclease activity, the TaqA289 DNA polymerase (TaqStoffel, KlenTaq) displays the increased thermostability, and a slightly increased requirement for Mg²⁺ ions, newly formed DNA strand contains fewer errors.

Research involving the obtaining of a DNA polymerase with deletion of consecutive amino acid residues from the N-end has revealed that the amino acid residues that are critical for optimal thermostability and activity are located in the region from 303 to 335. The crystallographic structure of the enzyme shows that the amino acid residues from this region form three β-sheets that interact with the rest of the enzyme.

Currently, PCR reactions show a very wide application in diagnostics, molecular biology, or genetic engineering. Their effectiveness is inextricably linked with the polymerase used, which is subjected to increasing demands connected with amplification of problematic DNA matrices. Therefore, it is necessary to search for DNA polymerases with new, useful features or improvement of those already used. The solution presented by us, i.e., Taq polymerase fused with DNA-binding protein, will allow to increase its affinity to DNA matrix, and thus will positively influence desirable features in diagnostics, i.e., processivity, efficiency, amplification of difficult matrices rich in GC, or DNA amplification from clinical samples allowing for significant acceleration of diagnostics in the case of many bacterial or viral diseases, and for obtaining reliable results.

The purpose of the invention is to create a new TaqPol polymerase with a NeqSSB protein that will be applicable to replicating specific sequences of the SARS CoV-2 virus.

The subject of the invention is a TaqPol-NeqSSB polymerase that binds all types of DNA and RNA. Three TaqPol-NeqSSB polymerase variants were subjected to the modifications:
TaqPol-NeqSSBFull—whole amino acid sequence of DNA I Taq polymerase with the whole amino acid sequence of NeqSSB protein (SEQ. ID 1),
TaqPol-NeqSSBII+III—DNA I Taq polymerase fused with domain II and III of NeqSSB (SEQ. ID 2),
TaqPol-NeqSSBIII—DNA I Taq polymerase fused with domain II and III of NeqSSB (SEQ. ID 3).

All variants of the TaqPol polymerase were fused with the NeqSSB protein by the polymerase C-end using a linker consisting of six amino acids (SEQ. ID 4).

The subject of the invention is a TaqPol-NeqSSB polymerase with SEQ. ID 1-3.

The subject of the invention is also a method for cloning a TaqPol-NeqSSB polymerase with SEQ.ID 1-3, which is used to obtain a DNA insert for cloning, which involves two independent PCR reactions:
the first amplification reaction yields a product with a nucleotide sequence corresponding to the gene sequence encoding the Taq DNA polymerase with an additional linker sequence and complementary to the 11 starting nucleotides of the NeqSSB protein at the C-end,
the second product contains the nucleotide sequence of the gene encoding the DNA-binding protein NeqSSB with additional nucleotides specific for the linker and 11 additional nucleotides complementary to the final nucleotide sequence of Taq polymerase at the N-end,
the isolated genomic DNA provides the template for the PCR reaction,
the products obtained in the two above-mentioned reactions are separated in agarose gel with ethidium bromide, and isolated from the gel.

The method, in which the products of two PCR reactions are used as inserts in a Gibson reaction, wherein:
Digestion of plasmid pET30EKLIC
To linearise the pET30EKLIC plasmid, it is digested with BamHI and NdeI (NEB) enzymes, which cut at two sites leaving the DNA ends non-complementary to each other,
The vector DNA digestion reaction is carried out for 2 h at 37° C. with addition of appropriate buffer,
The digested plasmid is separated electrophoretically and isolated,
Gene assembly reaction
The Gibson reaction is carried out in a thermocycler at 50° C. for 60 minutes, where the mixture contains buffer, nucleotides, enzymes, sterile water, Insert I, Insert II, vector,
After the reaction, the mixture is added to freshly prepared E. coli TOP10 competent cells,
The resulting mixture is incubated on ice for 40 min, after this incubation time a heat shock is performed by placing the cell mixture for 60 s in a 42° C. thermoblock, followed by 2 min of incubation on ice. After the heat shock, the cells are incubated for 60 min at 37° C. with 600 ml LB, then the cells are centrifuged (10 min, 1800 rpm), 500 ml of the filtrate was discarded, the pellet was resuspended in the remaining supernatant and seeded onto LA plates supplemented with kanamycin, the plates were incubated for approximately 16 h at 37° C.

A method where, to obtain a Taq-NeqSSB fusion protein, E. coli BL RIL cells are transformed using recombinant plasmid DNA pET30-TaqPol-NeqSSB, and production of the desired fusion protein is carried out. Cultures with the addition of kanamycin and chloramphenicol are grown for 16 h at 37° C., rejuvenated and when the cultures reach OD600=0.5, IPTG is added to a final concentration of 0.1 mM. After induction, the cultures are grown for another 5 h, after which they are centrifuged (10 min, 5000 rpm) and subjected to purification by metalloaffinity. The results of protein production are analysed by polyacrylamide electrophoresis of protein under denaturing conditions.

The subject of the invention is also an isolated recombinant plasmid that includes a fragment of the nucleotide sequence of the protein encoding the TaqPol-NeqSSB Full polymerase from 5076 to 8336 from the pET30EKLIC plasmid with SEQ. ID. 9, TaqPol-NeqSSBII+III from 5076 to 8159 from plasmid pET30EKLIC with SEQ. ID. 10 and TaqPol-NeqSSBIII from 5076 to 7886 from plasmid pET30EKLIC with SEQ. ID. 11.

The isolated plasmid pET30-TaqPol-NeqSSB Full with sequence SEQ.ID. 9, plasmid pET30-TaqPol-NeqSSBII+III with sequence SEQ.ID. 10, plasmid pET30-TaqPol-NeqSSBIII with sequence SEQ.ID. 11.

The subject of the invention also comprises primers for cloning TaqPol-NeqSSBFull/II+III/III polymerase with SEQ.ID sequences. 12.

The subject of the invention is a TaqPol-NeqSSBFull/II+III/III polymerase with SEQs. IDs 1, 2 and 3 to be applied in replicating specific sequences of SARS CoV-2 virus.

### DESCRIPTION OF THE FIGURES

FIG. 1—Schematic representation of NeqSSB protein with a basic OB domain

FIG. 2—represents Gibson assembly scheme

FIG. 3—represents an electrophoretic separation showing the results of purification of TaqPol-NeqSSBFul (A), TaqPol-NeqSSBII+III (B), TaqPol-NeqSSBIII (C) DNA polymerases on a His-Trap column
M—Protein mass marker (14,4-116 kDa) with the masses of the standard proteins: 116; 66,2; 45; 35; 25; 18,4; 14.4 kDa
1 —E. coli BL RIL/pET30-TaqPol-NeqSSBFull/II+III/III cell lysate after sonication and denaturation of host proteins
2—Fraction not bound with a column
3—Fraction after washing with buffer B (200 ml)
4—Fraction after elution with buffer C (30 ml)

FIG. 4—represents the electrophoretic separation in a 2% agarose gel with ethidium bromide showing the comparison of TaqPol-NeqSSBFull (A), TaqPol-NeqSSBII+III (B), TaqPol-NeqSSBIII (C) polymerase sensitivity in 10-fold serial dilutions of the template DNA

FIG. 5.—represents a diagram of the dependence of the SybrGreen fluorescence dye during RT qPCR reactions using TaqPol-NeqSSBFull (A), TaqPol-NeqSSBII+III (B), TaqPol-NeqSSBIII (C) polymerase in a reaction to identify SARS-CoV-2 virus directly from a swab.

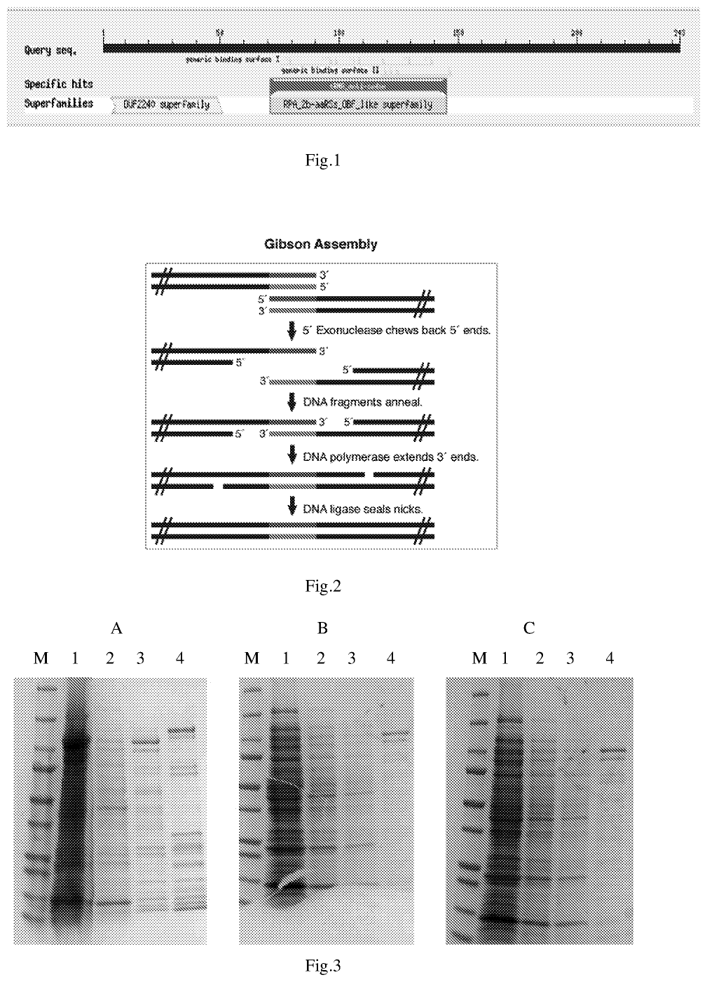

### DESCRIPTION OF THE SEQUENCES

SEQ. ID 1—represents the amino acid sequence of the TaqPol-NeqSSBFull fusion polymerase construct, where: Taq polymerase forms the main core of the polymerase, which will be fused to the NeqSSB protein (isolated from Nanoarchaeum equitans) at its C-end via a six-amino acid linker (GSGGVD). The presence of the linker gives the fusion protein a degree of flexibility and relatively free alignment with respect to the polymerase, in order to prevent possible steric hindrance.

SEQ.ID.2—represents the amino acid sequence of the TaqPol-NeqSSBII+III fusion polymerase construct, where: Taq polymerase forms the main core of the polymerase, which will be fused to the domains II and III of the NeqSSB protein (isolated from Nanoarchaeum equitans) at its C-end via a six-amino acid linker (GSGGVD). The presence of the linker gives the fusion protein a degree of flexibility and relatively free alignment with respect to the polymerase, in order to prevent possible steric hindrance.

SEQ.ID 3—represents the amino acid sequence of the TaqPol-NeqSSBIII fusion polymerase construct, where: Taq polymerase forms the main core of the polymerase, which will be fused to the domain III of the NeqSSB protein (isolated from Nanoarchaeum equitans) at its C-end via a six-amino acid linker (GSGGVD). The presence of the linker gives the fusion protein a degree of flexibility and relatively free alignment with respect to the polymerase, in order to prevent possible steric hindrance.

SEQ.ID. 4—represents the amino acid sequence of the linker SEQ.ID.5—represents the amino acid sequence of TaqPol polymerase SEQ.ID. 6—represents the amino acid sequence of the NeqSSB protein.

SEQ.ID. 7—represents the amino acid sequence of the domain II of the NeqSSB protein

SEQ.ID. 8—represents the amino acid sequence of the domain III of the NeqSSB protein

SEQ.ID. 9—represents the sequence of a plasmid carrying the gene encoding the TaqPol-NeqSSBFull protein

SEQ.ID. 10—represents the sequence of a plasmid carrying the gene encoding the TaqPol-NeqSSBII+III protein

SEQ.ID. 11—represents the sequence of a plasmid carrying the gene encoding the TaqPol-NeqSSBIII protein

SEQ.ID. 12—represents the primer sequences

The invention is illustrated by the following examples of implementation, which do not constitute a limitation of the invention

### Example 1

### I. Gene Encoding Polymerase, Expression Vector, Expression System

### a) Gene Encoding Polymerase: TaqPol-NeqSSBFull; TaqPol-NeqSSBII+III; TaqPol-NeqSSBIII

The amino acid sequence of TaqPol-NeqSSBFull/II+III/III polymerase (TaqPol-NeqSSBFull; TaqPol-NeqSSBII+III; TaqPol-NeqSSBIII) was extended by the sequence of the histidine domain necessary for efficient purification of the polymerase using the metal affinity. The 6×His domain was attached at the C-end of the polymerase. The nucleotide sequence of the polymerase (TaqPol-NeqSSBFull, TaqPol-NeqSSBII+III, TaqPol-NeqSSBIII) is shown on SEQ ID 1-3.

TaqPol-NeqSSB polymerase binds all types of DNA and RNA. Three variants of TaqPol-NeqSSB polymerase were modified:—TaqPol-NeqSSBFull—the entire amino acid sequence of Taq DNA polymerase I with the entire amino acid sequence of NeqSSB protein (SEQ. ID 1),
TaqPol-NeqSSBII+III—Taq DNA polymerase I in fusion with domains II and III of NeqSSB protein (SEQ. ID 2),
TaqPol-NeqSSBIII—Taq DNA polymerase I in fusion with domains II and III of NeqSSB protein (SEQ. ID 3). All variants of the TaqPol polymerase were fused with the NeqSSB protein by the polymerase C-end using a linker consisting of six amino acids (SEQ. ID 4).

### b) Expression Vector

The choice of vector for enzyme expression was based on the possibility of:
antibiotic selection of clones
cloning and its multiplication in bacterial cells
the presence of a promoter allowing easy control and induction of expression
   The pET30EKLIC vector carrying a kanamycin resistance gene, a bacterial origin of replication, and a T7 lactose promoter sequence allowing induction of expression by IPTG was selected. The vector has recognition sites for the restriction enzymes BamHI and NdeI, which cut the plasmid DNA at two sites giving two non-complementary sticky ends necessary for the cloning.

### c) Expression System

For the expression of thermostable fusion polymerase Pwo-NeqSSB, a prokaryotic system—E. coli, which is the most commonly used system for protein overproduction, both on laboratory and industrial scale, was chosen. IP-Free strains available from Promega: Escherichia coli BL21(DE3) pLysS were selected.

### d) Designing the SEQ.ID 12 Primers

A fusion polymerase consists of two proteins that are encoded by two independent genes. This forces the use of a cloning method that allows several DNA fragments to be cloned simultaneously. The Gibson method was used, which in a single reaction allows the generation of sticky ends (the 5′→3′ exonuclease), elongation of DNA ends (DNA polymerase), and covalent joining of two DNA ends (DNA ligase) of several fragments simultaneously. The OverLap kit (from A&A Biotechnology) was used. The cloning scheme is shown in FIG. 2.

### Example 2

### II. Cloning of Taq-NeqSSB Polymerase

### a) Amplification of Products for Cloning

Obtaining the insert DNA for cloning involved two independent PCR reactions. The first amplification reaction allowed to obtain a nucleotide sequence corresponding to the relevant DNA polymerase sequence with an additional linker sequence, and complementary to the 11 starting nucleotides of the NeqSSB protein at the C-end. The second product contained the nucleotide sequence of the DNA-binding protein with additional linker-specific nucleotides, and 11 additional nucleotides complementary to the final Taq polymerase nucleotide sequence at the N-end. The matrix for the PCR reaction was isolated genomic DNA. The products obtained in the above two reactions were separated in an agarose gel with ethidium bromide and isolated from the gel using the Gel-Out Concentrator kit (A&A Biotechnology). The products of these two PCR reactions were used as inserts in the Gibson reaction using the OverLap Assembly mix kit (A&A Biotechnology).
b) Digestion of Plasmid pET30EKLIC
To linearise the pET30EKLIC plasmid, it is digested with BamHI and NdeI (NEB) enzymes, which cut at two sites leaving the DNA ends non-complementary to each other. The vector DNA digestion reaction was carried out for 2 h at 37° C. with the addition of the appropriate buffer recommended by the manufacturer.
The digested plasmid was subjected to electrophoretic separation and isolated with a Gel-Out Concentrator kit (A&A Biotechnology).

### c) Gene Assembly Reaction

The Gibson reaction using the OverLap Assembly mix was run in a thermocycler at 50° C. for 60 minutes. The composition of the mixture is shown below:

| Component | Amount [ml] |
|---|---|
| Buffer 5 × OverLap Assembly | 4 |
| (A&A Biotechnology) | |
| Nucleotides [10 mM] | 2 |
| OverLap Assembly Enzimes | 2 |
| (A&A Biotechnology) | |
| Sterile water | 3 |
| Insert I [150 ng/ml] | 3 |
| Insert II [150 ng/ml] | 3 |
| Vector [150 ng/ml] | 3 |
| Final volume | 20 |

After reaction, the mixture was added to freshly prepared E. coli TOP10 competent cells.

### d) Transformation of Competent Cells

The Gibson reaction mixture was added to 100 ml of E. coli TOP10 competent cells. The resulting mixture was incubated on ice for 40 min. After this incubation time, a heat shock was performed by placing the cell mixture in a 42° C. thermoblock for 60 s, followed by 2 min of incubation on ice. After the heat shock, the cells were incubated for 60 min at 37° C. with 600 ml LB. After this time, the cells were centrifuged (10 min, 1800 rpm), 500 ml of the filtrate was discarded, the pellet was resuspended in the remaining supernatant and seeded onto LA plates supplemented with kanamycin. Plates were incubated for approximately 16 at 37° C.

### Example 3

### III. Expression and Purification of TaqPol-Neq Polymerase SSB

To obtain a Taq-NeqSSB fusion protein, E. coli BL RIL cells were transformed using recombinant plasmid DNA pET30-TaqPol-NeqSSBFull/II+III/III, and production of the desired fusion protein was carried out. Cultures with the addition of kanamycin and chloramphenicol were conducted for 16 hrs at 37° C., rejuvenated and when the cultures OD600=0.5, IPTG were added to a final concentration of 0.1 mM. After induction, the cultures were run for another 4 h, then they were centrifuged (10 min, 5000 rpm) and subjected to purification by metalloaffinity. The results of protein production were analysed by polyacrylamide electrophoresis of protein under denaturing conditions (SDS-PAGE) (FIG. 3). The molecular mass of the protein calculated using the Expasy computer program was 118.2 kDa:

TaqPol-NeqSSBFull:
Number of amino acids: 1080
Molecular mass: 121987,88
Theoretical pI value: 6,55

TaqPol-NeqSSBII+III:
Number of amino acids: 1021
Molecular mass: 115179,84
Theoretical pI value: 6,37

TaqPol-NeqSSBIII:
Number of amino acids: 930
Molecular mass: 104933,93
Theoretical pI value: 6,34

The results of the electrophoretic separations representing the degree of purification and the concentration of proteins after each stage of purification are shown in FIG. 3.

### Example 4

### IV. Activity and Application of the TaqPol-NeqSSB Polymerase

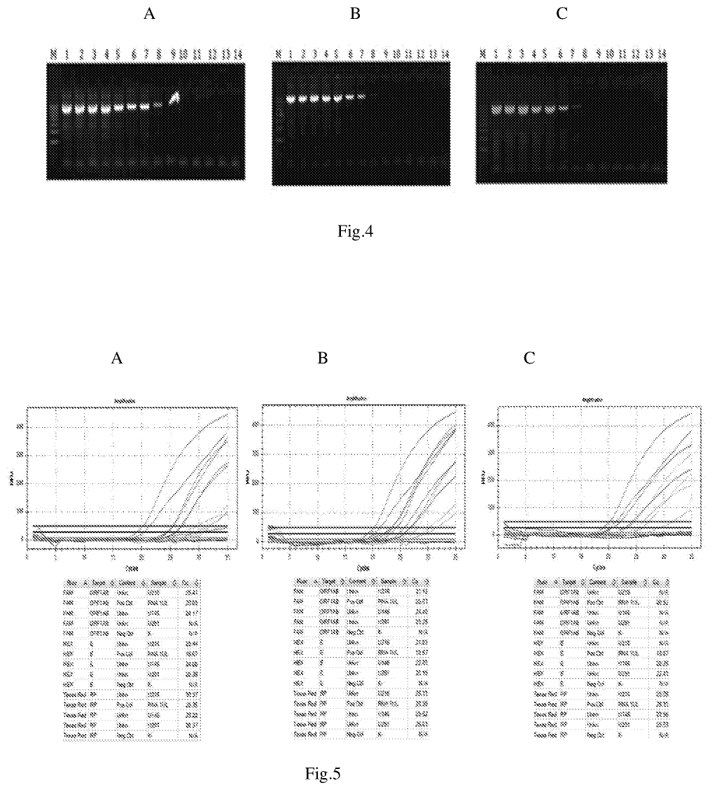

1) Application of the polymerase in a classical PCR reaction with agarose gel detection—plasmid DNA pUC19 target of 1000 bp in different concentrations of matrix DNA, (FIG. 4).
2) Application of polymerase in real-time RT-PCR reaction—identification of Orflab, E gene and human RP gene using different RNA matrices: total RNA of SARS-CoV-2 virus and directly from swab infected with SARS-CoV-2 virus, (FIG. 5).

### BIBLIOGRAPHY

[1] Vieille C, Burdette D S, Zeikus J G. Thermozymes. Biotechnol Annu Rev. 1996; 2:1-83.
[2] Hamilton S C, Farchaus J W, Davis M C. DNA polymerases as engines for biotechnology. Biotechniques. 2001; 31(2):370-6, 378-80, 382-3.
[3] Chien A, Edgar D B, Trela J M. Deoxyribonucleic acid polymerase from the extreme thermophile Thermus aquaticus. J Bacteriol. 1976; 127(3):1550-7.
[4] Vainshtein I, Atrazhev a, Eom S H, Elliott J F, Wishart D S, Malcolm B. Peptide rescue of an N-terminal truncation of the Stoffel fragment of Taq DNA polymerase. Protein Sci. 1996; 5(9):1785-92.
[5] Barnes W M. The fidelity of Taq polymerase catalyzing PCR is improved by an N-terminal deletion. Gene. 1992; 112(1):29-35.
[6] Rittié L, Perbal B. Enzymes used in molecular biology: a useful guide. J Cell Commun Signal. 2008; 2(1-2):25-45.
[7] Olszewski M, Balsewicz J, Nowak M, Maciejewska N, Cyranka-Czaja A, Zalewska-Piatek B, et al. Characterization of a Single-Stranded DNA-Binding-Like Protein from Nanoarchaeum equitans—Nucleic Acid Binding Protein with Broad Substrate Specificity. PLoS One. 2015; 10:e0126563.

SEQ ID 1
MRGMLPLFEPKGRVLLVDGHHLAYRTFHALKGLTTSRGEPVQAVYGFAKSLL
KALKEDGDAVIVVFDAKAPSFRHEAYGGYKAGRAPTPEDFPRQLALIKELVDL
LGLARLEVPGYEADDVLASLAKKAEKEGYEVRILTADKDLYQLLSDRIHALHP
EGYLITPAWLWEKYGLRPDQWADYRALTGDESDNLPGVKGIGEKTARKLLEE
WGSLEALLKNLDRLKPAIREKILAHMDDLKLSWDLAKVRTDLPLEVDFAKRRE
PDRERLRAFLERLEFGSLLHEFGLLESPKALEEAPWPPPEGAFVGFVLSRKEPM
WADLLALAAARGGRVHRAPEPYKALRDLKEARGLLAKDLSVLALREGLGLPP
GDDPMLLAYLLDPSNTTPEGVARRYGGEWTEEAGERAALSERLFANLWGRLE
GEERLLWLYREVERPLSAVLAHMEATGVRLDVAYLRALSLEVAEEIARLEAEV
FRLAGHPFNLNSRDQLERVLFDELGLPAIGKTEKTGKRSTSAAVLEALREAHPIV
EKILQYRELTKLKSTYIDPLPDLIHPRTGRLHTRFNQTATATGRLSSSDPNLQNIP
VRTPLGQRIRRAFIAEEGWLLVALDYSQIELRVLAHLSGDENLIRVFQEGRDIHT
ETASWMFGVPREAVDPLMRRAAKTINFGVLYGMSAHRLSQELAIPYEEAQAFI
ERYFQSFPKVRAWIEKTLEEGRRRGYVETLFGRRRYVPDLEARVKSVREAAER
MAFNMPVQGTAADLMKLAMVKLFPRLEEMGARMLLQVHDELVLEAPKERAE
AVARLAKEVMEGVYPLAVPLEVEVGIGEDWLSAKEGSGGVDDEEELIQLIIEKT
GKSREEIEKMVEEKIKAFNNLISRRGALLLVAKKLGVLYKNTPKEKKIGELESW
EYVKVKGKILKSFGLISYSKGKFQPIILGDETGTIKAIIWNTDKELPENTVIEAIGKTKINKKT
GNLELHIDSYKILESDLEIKPQKQEFVGICIVKYPKKQTQKGTIVSKAI
LTSLDRELPVVYFNDFDWEIGHIYKVYGKLKKNIKTGKIEFFADKVEEATLKDL
KAFKGEAD
SEQ. ID 2
MRGMLPLFEPKGRVLLVDGHHLAYRTFHALKGLTTSRGEPVQAVYGFAKSLL
KALKEDGDAVIVVFDAKAPSFRHEAYGGYKAGRAPTPEDFPRQLALIKELVDL
LGLARLEVPGYEADDVLASLAKKAEKEGYEVRILTADKDLYQLLSDRIHALHP
EGYLITPAWLWEKYGLRPDQWADYRALTGDESDNLPGVKGIGEKTARKLLEE
WGSLEALLKNLDRLKPAIREKILAHMDDLKLSWDLAKVRTDLPLEVDFAKRRE
PDRERLRAFLERLEFGSLLHEFGLLESPKALEEAPWPPPEGAFVGFVLSRKEPM
WADLLALAAARGGRVHRAPEPYKALRDLKEARGLLAKDLSVLALREGLGLPP
GDDPMLLAYLLDPSNTTPEGVARRYGGEWTEEAGERAALSERLFANLWGRLE
GEERLLWLYREVERPLSAVLAHMEATGVRLDVAYLRALSLEVAEEIARLEAEV
FRLAGHPFNLNSRDQLERVLFDELGLPAIGKTEKTGKRSTSAAVLEALREAHPIV
EKILQYRELTKLKSTYIDPLPDLIHPRTGRLHTRFNQTATATGRLSSSDPNLQNIP
VRTPLGQRIRRAFIAEEGWLLVALDYSQIELRVLAHLSGDENLIRVFQEGRDIHT
ETASWMFGVPREAVDPLMRRAAKTINFGVLYGMSAHRLSQELAIPYEEAQAFI
ERYFQSFPKVRAWIEKTLEEGRRRGYVETLFGRRRYVPDLEARVKSVREAAER
MAFNMPVQGTAADLMKLAMVKLFPRLEEMGARMLLQVHDELVLEAPKERAE
AVARLAKEVMEGVYPLAVPLEVEVGIGEDWLSAKEGSGGVDKIGELESWEYV
KVKGKILKSFGLISYSKGKFQPIILGDETGTIKAIIWNTDKELPENTVIEAIGKTKINKKTGNL
ELHIDSYKILESDLEIKPQKQEFVGICIVKYPKKQTQKGTIVSKAILTS
LDRELPVVYFNDFDWEIGHIYKVYGKLKKNIKTGKIEFFADKVEEATLKDLKAF
KGEAD
SEQ. ID 3
MRGMLPLFEPKGRVLLVDGHHLAYRTFHALKGLTTSRGEPVQAVYGFAKSLL
KALKEDGDAVIVVFDAKAPSFRHEAYGGYKAGRAPTPEDFPRQLALIKELVDL
LGLARLEVPGYEADDVLASLAKKAEKEGYEVRILTADKDLYQLLSDRIHALHP
EGYLITPAWLWEKYGLRPDQWADYRALTGDESDNLPGVKGIGEKTARKLLEE
WGSLEALLKNLDRLKPAIREKILAHMDDLKLSWDLAKVRTDLPLEVDFAKRRE
PDRERLRAFLERLEFGSLLHEFGLLESPKALEEAPWPPPEGAFVGFVLSRKEPM
WADLLALAAARGGRVHRAPEPYKALRDLKEARGLLAKDLSVLALREGLGLPP
GDDPMLLAYLLDPSNTTPEGVARRYGGEWTEEAGERAALSERLFANLWGRLE
GEERLLWLYREVERPLSAVLAHMEATGVRLDVAYLRALSLEVAEEIARLEAEV
FRLAGHPFNLNSRDQLERVLFDELGLPAIGKTEKTGKRSTSAAVLEALREAHPIV
EKILQYRELTKLKSTYIDPLPDLIHPRTGRLHTRFNQTATATGRLSSSDPNLQNIP
VRTPLGQRIRRAFIAEEGWLLVALDYSQIELRVLAHLSGDENLIRVFQEGRDIHT
ETASWMFGVPREAVDPLMRRAAKTINFGVLYGMSAHRLSQELAIPYEEAQAFI
ERYFQSFPKVRAWIEKTLEEGRRRGYVETLFGRRRYVPDLEARVKSVREAAER
MAFNMPVQGTAADLMKLAMVKLFPRLEEMGARMLLQVHDELVLEAPKERAE
AVARLAKEVMEGVYPLAVPLEVEVGIGEDWLSAKEGSGGVDKPQKQEFVGICI
VKYPKKQTQKGTIVSKAILTSLDRELPVVYFNDFDWEIGHIYKVYGKLKKNIKT
GKIEFFADKVEEATLKDLKAFKGEAD
SEQ. ID 4
GSGGVD
SEQ. ID 5
MRGMLPLFEPKGRVLLVDGHHLAYRTFHALKGLTTSRGEPVQAVYGFAKSLL
KALKEDGDAVIVVFDAKAPSFRHEAYGGYKAGRAPTPEDFPRQLALIKELVDL
LGLARLEVPGYEADDVLASLAKKAEKEGYEVRILTADKDLYQLLSDRIHALHP
EGYLITPAWLWEKYGLRPDQWADYRALTGDESDNLPGVKGIGEKTARKLLEE
WGSLEALLKNLDRLKPAIREKILAHMDDLKLSWDLAKVRTDLPLEVDFAKRRE
PDRERLRAFLERLEFGSLLHEFGLLESPKALEEAPWPPPEGAFVGFVLSRKEPM
WADLLALAAARGGRVHRAPEPYKALRDLKEARGLLAKDLSVLALREGLGLPP
GDDPMLLAYLLDPSNTTPEGVARRYGGEWTEEAGERAALSERLFANLWGRLE
GEERLLWLYREVERPLSAVLAHMEATGVRLDVAYLRALSLEVAEEIARLEAEV
FRLAGHPFNLNSRDQLERVLFDELGLPAIGKTEKTGKRSTSAAVLEALREAHPIV
EKILQYRELTKLKSTYIDPLPDLIHPRTGRLHTRFNQTATATGRLSSSDPNLQNIP
VRTPLGQRIRRAFIAEEGWLLVALDYSQIELRVLAHLSGDENLIRVFQEGRDIHT
ETASWMFGVPREAVDPLMRRAAKTINFGVLYGMSAHRLSQELAIPYEEAQAFI
ERYFQSFPKVRAWIEKTLEEGRRRGYVETLFGRRRYVPDLEARVKSVREAAER
MAFNMPVQGTAADLMKLAMVKLFPRLEEMGARMLLQVHDELVLEAPKERAE
AVARLAKEVMEGVYPLAVPLEVEVGIGEDWLSAKE
SEQ. ID 6
MDEEELIQLIIEKTGKSREEIEKMVEEKIKAFNNLISRRGALLLVAKKLGVLYKN
TPKEKKIGELESWEYVKVKGKILKSFGLISYSKGKFQPIILGDETGTIKAIIWNTD
KELPENTVIEAIGKTKINKKTGNLELHIDSYKILESDLEIKPQKQEFVGICIVKYPK
KQTQKGTIVSKAILTSLDRELPVVYFNDFDWEIGHIYKVYGKLKKNIKTGKIEFF
ADKVEEATLKDLKAFKGEAD
SEQ. ID 7
MKIGELESWEYVKVKGKILKSFGLISYSKGKFQPIILGDETGTIKAIIWNTDKELP
ENTVIEAIGKTKINKKTGNLELHIDSYKILESDLEI
SEQ. ID 8
MKPQKQEFVGICIVKYPKKQTQKGTIVSKAILTSLDRELPVVYFNDFDWEIGHIY
KVYGKLKKNIKTGKIEFFADKVEEATLKDLKAFKGEAD
SEQ. ID 9
TGGCGAATGGGACGCGCCCTGTAGCGGCGCATTAAGCGCGGCGGGTGTGGT
GGTTACGCGCAGCGTGACCGCTACACTTGCCAGCGCCCTAGCGCCCGCTCCT
TTCGCTTTCTTCCCTTCCTTTCTCGCCACGTTCGCCGGCTTTCCCCGTCAAGC
TCTAAATCGGGGGCTCCCTTTAGGGTTCCGATTTAGTGCTTTACGGCACCTC
GACCCCAAAAAACTTGATTAGGGTGATGGTTCACGTAGTGGGCCATCGCCC
TGATAGACGGTTTTTCGCCCTTTGACGTTGGAGTCCACGTTCTTTAATAGTG
GACTCTTGTTCCAAACTGGAACAACACTCAACCCTATCTCGGTCTATTCTTTT
GATTTATAAGGGATTTTGCCGATTTCGGCCTATTGGTTAAAAAATGAGCTGA
TTTAACAAAAATTTAACGCGAATTTTAACAAAATATTAACGTTTACAATTTC
AGGTGGCACTTTTCGGGGAAATGTGCGCGGAACCCCTATTTGTTTATTTTTC
TAAATACATTCAAATATGTATCCGCTCATGAATTAATTCTTAGAAAAACTCA
TCGAGCATCAAATGAAACTGCAATTTATTCATATCAGGATTATCAATACCAT
ATTTTTGAAAAAGCCGTTTCTGTAATGAAGGAGAAAACTCACCGAGGCAGT
TCCATAGGATGGCAAGATCCTGGTATCGGTCTGCGATTCCGACTCGTCCAAC
ATCAATACAACCTATTAATTTCCCCTCGTCAAAAATAAGGTTATCAAGTGAG
AAATCACCATGAGTGACGACTGAATCCGGTGAGAATGGCAAAAGTTTATGC
ATTTCTTTCCAGACTTGTTCAACAGGCCAGCCATTACGCTCGTCATCAAAAT
CACTCGCATCAACCAAACCGTTATTCATTCGTGATTGCGCCTGAGCGAGACG
AAATACGCGATCGCTGTTAAAAGGACAATTACAAACAGGAATCGAATGCAA
CCGGCGCAGGAACACTGCCAGCGCATCAACAATATTTTCACCTGAATCAGG
ATATTCTTCTAATACCTGGAATGCTGTTTTCCCGGGGATCGCAGTGGTGAGT
AACCATGCATCATCAGGAGTACGGATAAAATGCTTGATGGTCGGAAGAGGC
ATAAATTCCGTCAGCCAGTTTAGTCTGACCATCTCATCTGTAACATCATTGG
CAACGCTACCTTTGCCATGTTTCAGAAACAACTCTGGCGCATCGGGCTTCCC
ATACAATCGATAGATTGTCGCACCTGATTGCCCGACATTATCGCGAGCCCAT
TTATACCCATATAAATCAGCATCCATGTTGGAATTTAATCGCGGCCTAGAGC
AAGACGTTTCCCGTTGAATATGGCTCATAACACCCCTTGTATTACTGTTTAT
GTAAGCAGACAGTTTTATTGTTCATGACCAAAATCCCTTAACGTGAGTTTTC
GTTCCACTGAGCGTCAGACCCCGTAGAAAAGATCAAAGGATCTTCTTGAGA
TCCTTTTTTTCTGCGCGTAATCTGCTGCTTGCAAACAAAAAAACCACCGCTA
CCAGCGGTGGTTTGTTTGCCGGATCAAGAGCTACCAACTCTTTTTCCGAAGG
TAACTGGCTTCAGCAGAGCGCAGATACCAAATACTGTCCTTCTAGTGTAGCC
GTAGTTAGGCCACCACTTCAAGAACTCTGTAGCACCGCCTACATACCTCGCT
CTGCTAATCCTGTTACCAGTGGCTGCTGCCAGTGGCGATAAGTCGTGTCTTA
CCGGGTTGGACTCAAGACGATAGTTACCGGATAAGGCGCAGCGGTCGGGCT
GAACGGGGGGTTCGTGCACACAGCCCAGCTTGGAGCGAACGACCTACACCG
AACTGAGATACCTACAGCGTGAGCTATGAGAAAGCGCCACGCTTCCCGAAG
GGAGAAAGGCGGACAGGTATCCGGTAAGCGGCAGGGTCGGAACAGGAGAG
CGCACGAGGGAGCTTCCAGGGGGAAACGCCTGGTATCTTTATAGTCCTGTC
GGGTTTCGCCACCTCTGACTTGAGCGTCGATTTTTGTGATGCTCGTCAGGGG
GGCGGAGCCTATGGAAAAACGCCAGCAACGCGGCCTTTTTACGGTTCCTGG
CCTTTTGCTGGCCTTTTGCTCACATGTTCTTTCCTGCGTTATCCCCTGATTCTG
TGGATAACCGTATTACCGCCTTTGAGTGAGCTGATACCGCTCGCCGCAGCCG
AACGACCGAGCGCAGCGAGTCAGTGAGCGAGGAAGCGGAAGAGCGCCTGA
TGCGGTATTTTCTCCTTACGCATCTGTGCGGTATTTCACACCGCATATATGGT
GCACTCTCAGTACAATCTGCTCTGATGCCGCATAGTTAAGCCAGTATACACT
CCGCTATCGCTACGTGACTGGGTCATGGCTGCGCCCCGACACCCGCCAACA
CCCGCTGACGCGCCCTGACGGGCTTGTCTGCTCCCGGCATCCGCTTACAGAC
AAGCTGTGACCGTCTCCGGGAGCTGCATGTGTCAGAGGTTTTCACCGTCATC
ACCGAAACGCGCGAGGCAGCTGCGGTAAAGCTCATCAGCGTGGTCGTGAAG
CGATTCACAGATGTCTGCCTGTTCATCCGCGTCCAGCTCGTTGAGTTTCTCC
AGAAGCGTTAATGTCTGGCTTCTGATAAAGCGGGCCATGTTAAGGGCGGTT
TTTTCCTGTTTGGTCACTGATGCCTCCGTGTAAGGGGGATTTCTGTTCATGGG
GGTAATGATACCGATGAAACGAGAGAGGATGCTCACGATACGGGTTACTGA
TGATGAACATGCCCGGTTACTGGAACGTTGTGAGGGTAAACAACTGGCGGT
ATGGATGCGGCGGGACCAGAGAAAAATCACTCAGGGTCAATGCCAGCGCTT
CGTTAATACAGATGTAGGTGTTCCACAGGGTAGCCAGCAGCATCCTGCGAT
GCAGATCCGGAACATAATGGTGCAGGGCGCTGACTTCCGCGTTTCCAGACT
TTACGAAACACGGAAACCGAAGACCATTCATGTTGTTGCTCAGGTCGCAGA
CGTTTTGCAGCAGCAGTCGCTTCACGTTCGCTCGCGTATCGGTGATTCATTC
TGCTAACCAGTAAGGCAACCCCGCCAGCCTAGCCGGGTCCTCAACGACAGG
AGCACGATCATGCGCACCCGTGGGGCCGCCATGCCGGCGATAATGGCCTGC
TTCTCGCCGAAACGTTTGGTGGCGGGACCAGTGACGAAGGCTTGAGCGAGG
GCGTGCAAGATTCCGAATACCGCAAGCGACAGGCCGATCATCGTCGCGCTC
CAGCGAAAGCGGTCCTCGCCGAAAATGACCCAGAGCGCTGCCGGCACCTGT
CCTACGAGTTGCATGATAAAGAAGACAGTCATAAGTGCGGCGACGATAGTC
ATGCCCCGCGCCCACCGGAAGGAGCTGACTGGGTTGAAGGCTCTCAAGGGC
ATCGGTCGAGATCCCGGTGCCTAATGAGTGAGCTAACTTACATTAATTGCGT
TGCGCTCACTGCCCGCTTTCCAGTCGGGAAACCTGTCGTGCCAGCTGCATTA
ATGAATCGGCCAACGCGCGGGGAGAGGCGGTTTGCGTATTGGGCGCCAGGG
TGGTTTTTCTTTTCACCAGTGAGACGGGCAACAGCTGATTGCCCTTCACCGC
CTGGCCCTGAGAGAGTTGCAGCAAGCGGTCCACGCTGGTTTGCCCCAGCAG
GCGAAAATCCTGTTTGATGGTGGTTAACGGCGGGATATAACATGAGCTGTC
TTCGGTATCGTCGTATCCCACTACCGAGATGTCCGCACCAACGCGCAGCCCG
GACTCGGTAATGGCGCGCATTGCGCCCAGCGCCATCTGATCGTTGGCAACC
AGCATCGCAGTGGGAACGATGCCCTCATTCAGCATTTGCATGGTTTGTTGAA
AACCGGACATGGCACTCCAGTCGCCTTCCCGTTCCGCTATCGGCTGAATTTG
ATTGCGAGTGAGATATTTATGCCAGCCAGCCAGACGCAGACGCGCCGAGAC
AGAACTTAATGGGCCCGCTAACAGCGCGATTTGCTGGTGACCCAATGCGAC
CAGATGCTCCACGCCCAGTCGCGTACCGTCTTCATGGGAGAAAATAATACT
GTTGATGGGTGTCTGGTCAGAGACATCAAGAAATAACGCCGGAACATTAGT
GCAGGCAGCTTCCACAGCAATGGCATCCTGGTCATCCAGCGGATAGTTAAT
GATCAGCCCACTGACGCGTTGCGCGAGAAGATTGTGCACCGCCGCTTTACA
GGCTTCGACGCCGCTTCGTTCTACCATCGACACCACCACGCTGGCACCCAGT
TGATCGGCGCGAGATTTAATCGCCGCGACAATTTGCGACGGCGCGTGCAGG
GCCAGACTGGAGGTGGCAACGCCAATCAGCAACGACTGTTTGCCCGCCAGT
TGTTGTGCCACGCGGTTGGGAATGTAATTCAGCTCCGCCATCGCCGCTTCCA
CTTTTTCCCGCGTTTTCGCAGAAACGTGGCTGGCCTGGTTCACCACGCGGGA
AACGGTCTGATAAGAGACACCGGCATACTCTGCGACATCGTATAACGTTAC
TGGTTTCACATTCACCACCCTGAATTGACTCTCTTCCGGGCGCTATCATGCC
ATACCGCGAAAGGTTTTGCGCCATTCGATGGTGTCCGGGATCTCGACGCTCT
CCCTTATGCGACTCCTGCATTAGGAAGCAGCCCAGTAGTAGGTTGAGGCCG
TTGAGCACCGCCGCCGCAAGGAATGGTGCATGCAAGGAGATGGCGCCCAAC
AGTCCCCCGGCCACGGGGCCTGCCACCATACCCACGCCGAAACAAGCGCTC
ATGAGCCCGAAGTGGCGAGCCCGATCTTCCCCATCGGTGATGTCGGCGATA
TAGGCGCCAGCAACCGCACCTGTGGCGCCGGTGATGCCGGCCACGATGCGT
CCGGCGTAGAGGATCGAGATCGATCTCGATCCCGCGAAATTAATACGACTC
ACTATAGGGGAATTGTGAGCGGATAACAATTCCCCTCTAGAAATAATTTTGT
TTAACTTTAAGAAGGAGATATACATATGAGGGGGATGCTGCCCCTCTTTGA
GCCCAAGGGCCGGGTCCTCCTGGTGGACGGCCACCACCTGGCCTACCGCAC
CTTCCACGCCCTGAAGGGCCTCACCACCAGCCGGGGGGAGCCGGTGCAGGC
GGTCTACGGCTTCGCCAAGAGCCTCCTCAAGGCCCTCAAGGAGGACGGGGA
CGCGGTGATCGTGGTCTTTGACGCCAAGGCCCCCTCCTTCCGCCACGAGGCC
TACGGGGGGTACAAGGCGGGCCGGGCCCCCACGCCGGAGGACTTTCCCCGG
CAACTCGCCCTCATCAAGGAGCTGGTGGACCTCCTGGGGCTGGCGCGCCTC
GAGGTCCCGGGCTACGAGGCGGACGACGTCCTGGCCAGCCTGGCCAAGAAG
GCGGAAAAGGAGGGCTACGAGGTCCGCATCCTCACCGCCGACAAAGACCTT
TACCAGCTCCTTTCCGACCGCATCCACGCCCTCCACCCCGAGGGGTACCTCA
TCACCCCGGCCTGGCTTTGGGAAAAGTACGGCCTGAGGCCCGACCAGTGGG
CCGACTACCGGGCCCTGACCGGGGACGAGTCCGACAACCTTCCCGGGGTCA
AGGGCATCGGGGAGAAGACGGCGAGGAAGCTTCTGGAGGAGTGGGGGAGC
CTGGAAGCCCTCCTCAAGAACCTGGACCGGCTGAAGCCCGCCATCCGGGAG
AAGATCCTGGCCCACATGGACGATCTGAAGCTCTCCTGGGACCTGGCCAAG
GTGCGCACCGACCTGCCCCTGGAGGTGGACTTCGCCAAAAGGCGGGAGCCC
GACCGGGAGAGGCTTAGGGCCTTTCTGGAGAGGCTTGAGTTTGGCAGCCTC
CTCCACGAGTTCGGCCTTCTGGAAAGCCCCAAGGCCCTGGAGGAGGCCCCC
TGGCCCCCGCCGGAAGGGGCCTTCGTGGGCTTTGTGCTTTCCCGCAAGGAGC
CCATGTGGGCCGATCTTCTGGCCCTGGCCGCCGCCAGGGGGGGCCGGGTCC
ACCGGGCCCCCGAGCCTTATAAAGCCCTCAGGGACCTGAAGGAGGCGCGGG
GGCTTCTCGCCAAAGACCTGAGCGTTCTGGCCCTGAGGGAAGGCCTTGGCC
TCCCGCCCGGCGACGACCCCATGCTCCTCGCCTACCTCCTGGACCCTTCCAA
CACCACCCCCGAGGGGGTGGCCCGGCGCTACGGCGGGGAGTGGACGGAGG
AGGCGGGGGAGCGGGCCGCCCTTTCCGAGAGGCTCTTCGCCAACCTGTGGG
GGAGGCTTGAGGGGGAGGAGAGGCTCCTTTGGCTTTACCGGGAGGTGGAGA
GGCCCCTTTCCGCTGTCCTGGCCCACATGGAGGCCACGGGGGTGCGCCTGG
ACGTGGCCTATCTCAGGGCCTTGTCCCTGGAGGTGGCCGAGGAGATCGCCC
GCCTCGAGGCCGAGGTCTTCCGCCTGGCCGGCCACCCCTTCAACCTCAACTC
CCGGGACCAGCTGGAAAGGGTCCTCTTTGACGAGCTAGGGCTTCCCGCCAT
CGGCAAGACGGAGAAGACCGGCAAGCGCTCCACCAGCGCCGCCGTCCTGG
AGGCCCTCCGCGAGGCCCACCCCATCGTGGAGAAGATCCTGCAGTACCGGG
AGCTCACCAAGCTGAAGAGCACCTACATTGACCCCTTGCCGGACCTCATCC
ACCCCAGGACGGGCCGCCTCCACACCCGCTTCAACCAGACGGCCACGGCCA
CGGGCAGGCTAAGTAGCTCCGATCCCAACCTCCAGAACATCCCCGTCCGCA
CCCCGCTTGGGCAGAGGATCCGCCGGGCCTTCATCGCCGAGGAGGGGTGGC
TATTGGTGGCCCTGGACTATAGCCAGATAGAGCTCAGGGTGCTGGCCCACC
TCTCCGGCGACGAGAACCTGATCCGGGTCTTCCAGGAGGGGCGGGACATCC
ACACGGAGACCGCCAGCTGGATGTTCGGCGTCCCCCGGGAGGCCGTGGACC
CCCTGATGCGCCGGGCGGCCAAGACCATCAACTTCGGGGTCCTCTACGGCA
TGTCGGCCCACCGCCTCTCCCAGGAGCTAGCCATCCCTTACGAGGAGGCCC
AGGCCTTCATTGAGCGCTACTTTCAGAGCTTCCCCAAGGTGCGGGCCTGGAT
TGAGAAGACCCTGGAGGAGGGCAGGAGGCGGGGGTACGTGGAGACCCTCT
TCGGCCGCCGCCGCTACGTGCCAGACCTAGAGGCCCGGGTGAAGAGCGTGC
GGGAGGCGGCCGAGCGCATGGCCTTCAACATGCCCGTCCAGGGCACCGCCG
CCGACCTCATGAAGCTGGCTATGGTGAAGCTCTTCCCCAGGCTGGAGGAAA
TGGGGGCCAGGATGCTCCTTCAGGTCCACGACGAGCTGGTCCTCGAGGCCC
CAAAAGAGAGGGCGGAGGCCGTGGCCCGGCTGGCCAAGGAGGTCATGGAG
GGGGTGTATCCCCTGGCCGTGCCCCTGGAGGTGGAGGTGGGGATAGGGGAG
GACTGGCTCTCCGCCAAGGAGGGCAGCGGTGGCGTTGATGATGAAGAGGAA
CTAATACAACTAATAATAGAAAAAACTGGCAAATCTCGAGAGGAAATAGA
AAAAATGGTGGAAGAAAAAATTAAAGCTTTTAACAATTTAATATCTCGTAG
GGGGGCTTTACTATTAGTAGCAAAAAAACTTGGTGTTTTGTATAAAAACACT
CCGAAAGAGAAAAAAATTGGCGAATTAGAAAGCTGGGAATATGTAAAAGT
AAAGGGCAAAATTCTCAAATCTTTTGGATTAATTAGTTATTCGAAAGGGAA
ATTCCAACCTATTATTTTAGGAGACGAAACCGGTACTATTAAAGCTATTATT
TGGAATACCGATAAAGAATTACCTGAAAACACTGTAATAGAAGCTATTGGG
AAAACCAAAATTAATAAGAAAACTGGCAATTTAGAATTACATATAGACAGT
TATAAAATTTTAGAAAGCGATTTAGAGATAAAACCCCAAAAGCAAGAATTT
GTTGGGATTTGCATAGTTAAATATCCAAAAAAACAAACCCAAAAAGGCACA
ATAGTATCGAAAGCAATTTTAACTAGCTTAGATAGGGAATTGCCTGTAGTAT
ATTTCAACGATTTTGATTGGGAAATAGGCCATATATATAAAGTATATGGAA
AGCTTAAGAAAAACATAAAAACTGGTAAAATAGAATTTTTCGCTGACAAAG
TTGAGGAAGCAACATTAAAAGATCTAAAAGCTTTTAAAGGAGAGGCCGATC
ACCACCACCACCACCACTAAGGATCCGAATTCGAGCTCCGTCGACAAGCTT
GCGGCCGCACTCGAGCACCACCACCACCACCACTGAGATCCGGCTGCTAAC
AAAGCCCGAAAGGAAGCTGAGTTGGCTGCTGCCACCGCTGAGCAATAACTA
GCATAACCCCTTGGGGCCTCTAAACGGGTCTTGAGGGGTTTTTTGCTGAAAG
GAGGAACTATATCCGGAT
SEQ. ID 10
TGGCGAATGGGACGCGCCCTGTAGCGGCGCATTAAGCGCGGCGGGTGTGGT
GGTTACGCGCAGCGTGACCGCTACACTTGCCAGCGCCCTAGCGCCCGCTCCT
TTCGCTTTCTTCCCTTCCTTTCTCGCCACGTTCGCCGGCTTTCCCCGTCAAGC
TCTAAATCGGGGGCTCCCTTTAGGGTTCCGATTTAGTGCTTTACGGCACCTC
GACCCCAAAAAACTTGATTAGGGTGATGGTTCACGTAGTGGGCCATCGCCC
TGATAGACGGTTTTTCGCCCTTTGACGTTGGAGTCCACGTTCTTTAATAGTG
GACTCTTGTTCCAAACTGGAACAACACTCAACCCTATCTCGGTCTATTCTTTT
GATTTATAAGGGATTTTGCCGATTTCGGCCTATTGGTTAAAAAATGAGCTGA
TTTAACAAAAATTTAACGCGAATTTTAACAAAATATTAACGTTTACAATTTC
AGGTGGCACTTTTCGGGGAAATGTGCGCGGAACCCCTATTTGTTTATTTTTC
TAAATACATTCAAATATGTATCCGCTCATGAATTAATTCTTAGAAAAACTCA
TCGAGCATCAAATGAAACTGCAATTTATTCATATCAGGATTATCAATACCAT
ATTTTTGAAAAAGCCGTTTCTGTAATGAAGGAGAAAACTCACCGAGGCAGT
TCCATAGGATGGCAAGATCCTGGTATCGGTCTGCGATTCCGACTCGTCCAAC
ATCAATACAACCTATTAATTTCCCCTCGTCAAAAATAAGGTTATCAAGTGAG
AAATCACCATGAGTGACGACTGAATCCGGTGAGAATGGCAAAAGTTTATGC
ATTTCTTTCCAGACTTGTTCAACAGGCCAGCCATTACGCTCGTCATCAAAAT
CACTCGCATCAACCAAACCGTTATTCATTCGTGATTGCGCCTGAGCGAGACG
AAATACGCGATCGCTGTTAAAAGGACAATTACAAACAGGAATCGAATGCAA
CCGGCGCAGGAACACTGCCAGCGCATCAACAATATTTTCACCTGAATCAGG
ATATTCTTCTAATACCTGGAATGCTGTTTTCCCGGGGATCGCAGTGGTGAGT
AACCATGCATCATCAGGAGTACGGATAAAATGCTTGATGGTCGGAAGAGGC
ATAAATTCCGTCAGCCAGTTTAGTCTGACCATCTCATCTGTAACATCATTGG
CAACGCTACCTTTGCCATGTTTCAGAAACAACTCTGGCGCATCGGGCTTCCC
ATACAATCGATAGATTGTCGCACCTGATTGCCCGACATTATCGCGAGCCCAT
TTATACCCATATAAATCAGCATCCATGTTGGAATTTAATCGCGGCCTAGAGC
AAGACGTTTCCCGTTGAATATGGCTCATAACACCCCTTGTATTACTGTTTAT
GTAAGCAGACAGTTTTATTGTTCATGACCAAAATCCCTTAACGTGAGTTTTC
GTTCCACTGAGCGTCAGACCCCGTAGAAAAGATCAAAGGATCTTCTTGAGA
TCCTTTTTTTCTGCGCGTAATCTGCTGCTTGCAAACAAAAAAACCACCGCTA
CCAGCGGTGGTTTGTTTGCCGGATCAAGAGCTACCAACTCTTTTTCCGAAGG
TAACTGGCTTCAGCAGAGCGCAGATACCAAATACTGTCCTTCTAGTGTAGCC
GTAGTTAGGCCACCACTTCAAGAACTCTGTAGCACCGCCTACATACCTCGCT
CTGCTAATCCTGTTACCAGTGGCTGCTGCCAGTGGCGATAAGTCGTGTCTTA
CCGGGTTGGACTCAAGACGATAGTTACCGGATAAGGCGCAGCGGTCGGGCT
GAACGGGGGGTTCGTGCACACAGCCCAGCTTGGAGCGAACGACCTACACCG
AACTGAGATACCTACAGCGTGAGCTATGAGAAAGCGCCACGCTTCCCGAAG
GGAGAAAGGCGGACAGGTATCCGGTAAGCGGCAGGGTCGGAACAGGAGAG
CGCACGAGGGAGCTTCCAGGGGGAAACGCCTGGTATCTTTATAGTCCTGTC
GGGTTTCGCCACCTCTGACTTGAGCGTCGATTTTTGTGATGCTCGTCAGGGG
GGCGGAGCCTATGGAAAAACGCCAGCAACGCGGCCTTTTTACGGTTCCTGG
CCTTTTGCTGGCCTTTTGCTCACATGTTCTTTCCTGCGTTATCCCCTGATTCTG
TGGATAACCGTATTACCGCCTTTGAGTGAGCTGATACCGCTCGCCGCAGCCG
AACGACCGAGCGCAGCGAGTCAGTGAGCGAGGAAGCGGAAGAGCGCCTGA
TGCGGTATTTTCTCCTTACGCATCTGTGCGGTATTTCACACCGCATATATGGT
GCACTCTCAGTACAATCTGCTCTGATGCCGCATAGTTAAGCCAGTATACACT
CCGCTATCGCTACGTGACTGGGTCATGGCTGCGCCCCGACACCCGCCAACA
CCCGCTGACGCGCCCTGACGGGCTTGTCTGCTCCCGGCATCCGCTTACAGAC
AAGCTGTGACCGTCTCCGGGAGCTGCATGTGTCAGAGGTTTTCACCGTCATC
ACCGAAACGCGCGAGGCAGCTGCGGTAAAGCTCATCAGCGTGGTCGTGAAG
CGATTCACAGATGTCTGCCTGTTCATCCGCGTCCAGCTCGTTGAGTTTCTCC
AGAAGCGTTAATGTCTGGCTTCTGATAAAGCGGGCCATGTTAAGGGCGGTT
TTTTCCTGTTTGGTCACTGATGCCTCCGTGTAAGGGGGATTTCTGTTCATGGG
GGTAATGATACCGATGAAACGAGAGAGGATGCTCACGATACGGGTTACTGA
TGATGAACATGCCCGGTTACTGGAACGTTGTGAGGGTAAACAACTGGCGGT
ATGGATGCGGCGGGACCAGAGAAAAATCACTCAGGGTCAATGCCAGCGCTT
CGTTAATACAGATGTAGGTGTTCCACAGGGTAGCCAGCAGCATCCTGCGAT
GCAGATCCGGAACATAATGGTGCAGGGCGCTGACTTCCGCGTTTCCAGACT
TTACGAAACACGGAAACCGAAGACCATTCATGTTGTTGCTCAGGTCGCAGA
CGTTTTGCAGCAGCAGTCGCTTCACGTTCGCTCGCGTATCGGTGATTCATTC
TGCTAACCAGTAAGGCAACCCCGCCAGCCTAGCCGGGTCCTCAACGACAGG
AGCACGATCATGCGCACCCGTGGGGCCGCCATGCCGGCGATAATGGCCTGC
TTCTCGCCGAAACGTTTGGTGGCGGGACCAGTGACGAAGGCTTGAGCGAGG
GCGTGCAAGATTCCGAATACCGCAAGCGACAGGCCGATCATCGTCGCGCTC
CAGCGAAAGCGGTCCTCGCCGAAAATGACCCAGAGCGCTGCCGGCACCTGT
CCTACGAGTTGCATGATAAAGAAGACAGTCATAAGTGCGGCGACGATAGTC
ATGCCCCGCGCCCACCGGAAGGAGCTGACTGGGTTGAAGGCTCTCAAGGGC
ATCGGTCGAGATCCCGGTGCCTAATGAGTGAGCTAACTTACATTAATTGCGT
TGCGCTCACTGCCCGCTTTCCAGTCGGGAAACCTGTCGTGCCAGCTGCATTA
ATGAATCGGCCAACGCGCGGGGAGAGGCGGTTTGCGTATTGGGCGCCAGGG
TGGTTTTTCTTTTCACCAGTGAGACGGGCAACAGCTGATTGCCCTTCACCGC
CTGGCCCTGAGAGAGTTGCAGCAAGCGGTCCACGCTGGTTTGCCCCAGCAG
GCGAAAATCCTGTTTGATGGTGGTTAACGGCGGGATATAACATGAGCTGTC
TTCGGTATCGTCGTATCCCACTACCGAGATGTCCGCACCAACGCGCAGCCCG
GACTCGGTAATGGCGCGCATTGCGCCCAGCGCCATCTGATCGTTGGCAACC
AGCATCGCAGTGGGAACGATGCCCTCATTCAGCATTTGCATGGTTTGTTGAA
AACCGGACATGGCACTCCAGTCGCCTTCCCGTTCCGCTATCGGCTGAATTTG
ATTGCGAGTGAGATATTTATGCCAGCCAGCCAGACGCAGACGCGCCGAGAC
AGAACTTAATGGGCCCGCTAACAGCGCGATTTGCTGGTGACCCAATGCGAC
CAGATGCTCCACGCCCAGTCGCGTACCGTCTTCATGGGAGAAAATAATACT
GTTGATGGGTGTCTGGTCAGAGACATCAAGAAATAACGCCGGAACATTAGT
GCAGGCAGCTTCCACAGCAATGGCATCCTGGTCATCCAGCGGATAGTTAAT
GATCAGCCCACTGACGCGTTGCGCGAGAAGATTGTGCACCGCCGCTTTACA
GGCTTCGACGCCGCTTCGTTCTACCATCGACACCACCACGCTGGCACCCAGT
TGATCGGCGCGAGATTTAATCGCCGCGACAATTTGCGACGGCGCGTGCAGG
GCCAGACTGGAGGTGGCAACGCCAATCAGCAACGACTGTTTGCCCGCCAGT
TGTTGTGCCACGCGGTTGGGAATGTAATTCAGCTCCGCCATCGCCGCTTCCA
CTTTTTCCCGCGTTTTCGCAGAAACGTGGCTGGCCTGGTTCACCACGCGGGA
AACGGTCTGATAAGAGACACCGGCATACTCTGCGACATCGTATAACGTTAC
TGGTTTCACATTCACCACCCTGAATTGACTCTCTTCCGGGCGCTATCATGCC
ATACCGCGAAAGGTTTTGCGCCATTCGATGGTGTCCGGGATCTCGACGCTCT
CCCTTATGCGACTCCTGCATTAGGAAGCAGCCCAGTAGTAGGTTGAGGCCG
TTGAGCACCGCCGCCGCAAGGAATGGTGCATGCAAGGAGATGGCGCCCAAC
AGTCCCCCGGCCACGGGGCCTGCCACCATACCCACGCCGAAACAAGCGCTC
ATGAGCCCGAAGTGGCGAGCCCGATCTTCCCCATCGGTGATGTCGGCGATA
TAGGCGCCAGCAACCGCACCTGTGGCGCCGGTGATGCCGGCCACGATGCGT
CCGGCGTAGAGGATCGAGATCGATCTCGATCCCGCGAAATTAATACGACTC
ACTATAGGGGAATTGTGAGCGGATAACAATTCCCCTCTAGAAATAATTTTGT
TTAACTTTAAGAAGGAGATATACATATGAGGGGGATGCTGCCCCTCTTTGA
GCCCAAGGGCCGGGTCCTCCTGGTGGACGGCCACCACCTGGCCTACCGCAC
CTTCCACGCCCTGAAGGGCCTCACCACCAGCCGGGGGGAGCCGGTGCAGGC
GGTCTACGGCTTCGCCAAGAGCCTCCTCAAGGCCCTCAAGGAGGACGGGGA
CGCGGTGATCGTGGTCTTTGACGCCAAGGCCCCCTCCTTCCGCCACGAGGCC
TACGGGGGGTACAAGGCGGGCCGGGCCCCCACGCCGGAGGACTTTCCCCGG
CAACTCGCCCTCATCAAGGAGCTGGTGGACCTCCTGGGGCTGGCGCGCCTC
GAGGTCCCGGGCTACGAGGCGGACGACGTCCTGGCCAGCCTGGCCAAGAAG
GCGGAAAAGGAGGGCTACGAGGTCCGCATCCTCACCGCCGACAAAGACCTT
TACCAGCTCCTTTCCGACCGCATCCACGCCCTCCACCCCGAGGGGTACCTCA
TCACCCCGGCCTGGCTTTGGGAAAAGTACGGCCTGAGGCCCGACCAGTGGG
CCGACTACCGGGCCCTGACCGGGGACGAGTCCGACAACCTTCCCGGGGTCA
AGGGCATCGGGGAGAAGACGGCGAGGAAGCTTCTGGAGGAGTGGGGGAGC
CTGGAAGCCCTCCTCAAGAACCTGGACCGGCTGAAGCCCGCCATCCGGGAG
AAGATCCTGGCCCACATGGACGATCTGAAGCTCTCCTGGGACCTGGCCAAG
GTGCGCACCGACCTGCCCCTGGAGGTGGACTTCGCCAAAAGGCGGGAGCCC
GACCGGGAGAGGCTTAGGGCCTTTCTGGAGAGGCTTGAGTTTGGCAGCCTC
CTCCACGAGTTCGGCCTTCTGGAAAGCCCCAAGGCCCTGGAGGAGGCCCCC
TGGCCCCCGCCGGAAGGGGCCTTCGTGGGCTTTGTGCTTTCCCGCAAGGAGC
CCATGTGGGCCGATCTTCTGGCCCTGGCCGCCGCCAGGGGGGGCCGGGTCC
ACCGGGCCCCCGAGCCTTATAAAGCCCTCAGGGACCTGAAGGAGGCGCGGG
GGCTTCTCGCCAAAGACCTGAGCGTTCTGGCCCTGAGGGAAGGCCTTGGCC
TCCCGCCCGGCGACGACCCCATGCTCCTCGCCTACCTCCTGGACCCTTCCAA
CACCACCCCCGAGGGGGTGGCCCGGCGCTACGGGGGGAGTGGACGGAGG
AGGCGGGGGAGCGGGCCGCCCTTTCCGAGAGGCTCTTCGCCAACCTGTGGG
GGAGGCTTGAGGGGGAGGAGAGGCTCCTTTGGCTTTACCGGGAGGTGGAGA
GGCCCCTTTCCGCTGTCCTGGCCCACATGGAGGCCACGGGGGTGCGCCTGG
ACGTGGCCTATCTCAGGGCCTTGTCCCTGGAGGTGGCCGAGGAGATCGCCC
GCCTCGAGGCCGAGGTCTTCCGCCTGGCCGGCCACCCCTTCAACCTCAACTC
CCGGGACCAGCTGGAAAGGGTCCTCTTTGACGAGCTAGGGCTTCCCGCCAT
CGGCAAGACGGAGAAGACCGGCAAGCGCTCCACCAGCGCCGCCGTCCTGG
AGGCCCTCCGCGAGGCCCACCCCATCGTGGAGAAGATCCTGCAGTACCGGG
AGCTCACCAAGCTGAAGAGCACCTACATTGACCCCTTGCCGGACCTCATCC
ACCCCAGGACGGGCCGCCTCCACACCCGCTTCAACCAGACGGCCACGGCCA
CGGGCAGGCTAAGTAGCTCCGATCCCAACCTCCAGAACATCCCCGTCCGCA
CCCCGCTTGGGCAGAGGATCCGCCGGGCCTTCATCGCCGAGGAGGGGTGGC
TATTGGTGGCCCTGGACTATAGCCAGATAGAGCTCAGGGTGCTGGCCCACC
TCTCCGGCGACGAGAACCTGATCCGGGTCTTCCAGGAGGGGGGGGACATCC
ACACGGAGACCGCCAGCTGGATGTTCGGCGTCCCCCGGGAGGCCGTGGACC
CCCTGATGCGCCGGGCGGCCAAGACCATCAACTTCGGGGTCCTCTACGGCA
TGTCGGCCCACCGCCTCTCCCAGGAGCTAGCCATCCCTTACGAGGAGGCCC
AGGCCTTCATTGAGCGCTACTTTCAGAGCTTCCCCAAGGTGCGGGCCTGGAT
TGAGAAGACCCTGGAGGAGGGCAGGAGGCGGGGGTACGTGGAGACCCTCT
TCGGCCGCCGCCGCTACGTGCCAGACCTAGAGGCCCGGGTGAAGAGCGTGC
GGGAGGCGGCCGAGCGCATGGCCTTCAACATGCCCGTCCAGGGCACCGCCG
CCGACCTCATGAAGCTGGCTATGGTGAAGCTCTTCCCCAGGCTGGAGGAAA
TGGGGGCCAGGATGCTCCTTCAGGTCCACGACGAGCTGGTCCTCGAGGCCC
CAAAAGAGAGGGCGGAGGCCGTGGCCCGGCTGGCCAAGGAGGTCATGGAG
GGGGTGTATCCCCTGGCCGTGCCCCTGGAGGTGGAGGTGGGGATAGGGGAG
GACTGGCTCTCCGCCAAGGAGGGCAGCGGTGGCGTTGATAAAATTGGCGAA
TTAGAAAGCTGGGAATATGTAAAAGTAAAGGGCAAAATTCTCAAATCTTTT
GGATTAATTAGTTATTCGAAAGGGAAATTCCAACCTATTATTTTAGGAGACG
AAACCGGTACTATTAAAGCTATTATTTGGAATACCGATAAAGAATTACCTG
AAAACACTGTAATAGAAGCTATTGGGAAAACCAAAATTAATAAGAAAACTG
GCAATTTAGAATTACATATAGACAGTTATAAAATTTTAGAAAGCGATTTAG
AGATAAAACCCCAAAAGCAAGAATTTGTTGGGATTTGCATAGTTAAATATC
CAAAAAAACAAACCCAAAAAGGCACAATAGTATCGAAAGCAATTTTAACTA
GCTTAGATAGGGAATTGCCTGTAGTATATTTCAACGATTTTGATTGGGAAAT
AGGCCATATATATAAAGTATATGGAAAGCTTAAGAAAAACATAAAAACTGG
TAAAATAGAATTTTTCGCTGACAAAGTTGAGGAAGCAACATTAAAAGATCT
AAAAGCTTTTAAAGGAGAGGCCGATCACCACCACCACCACCACTAAGGATC
CGAATTCGAGCTCCGTCGACAAGCTTGCGGCCGCACTCGAGCACCACCACC
ACCACCACTGAGATCCGGCTGCTAACAAAGCCCGAAAGGAAGCTGAGTTGG
CTGCTGCCACCGCTGAGCAATAACTAGCATAACCCCTTGGGGCCTCTAAAC
GGGTCTTGAGGGGTTTTTTGCTGAAAGGAGGAACTATATCCGGAT
SEQ. ID 11
TGGCGAATGGGACGCGCCCTGTAGCGGCGCATTAAGCGCGGCGGGTGTGGT
GGTTACGCGCAGCGTGACCGCTACACTTGCCAGCGCCCTAGCGCCCGCTCCT
TTCGCTTTCTTCCCTTCCTTTCTCGCCACGTTCGCCGGCTTTCCCCGTCAAGC
TCTAAATCGGGGGCTCCCTTTAGGGTTCCGATTTAGTGCTTTACGGCACCTC
GACCCCAAAAAACTTGATTAGGGTGATGGTTCACGTAGTGGGCCATCGCCC
TGATAGACGGTTTTTCGCCCTTTGACGTTGGAGTCCACGTTCTTTAATAGTG
GACTCTTGTTCCAAACTGGAACAACACTCAACCCTATCTCGGTCTATTCTTTT
GATTTATAAGGGATTTTGCCGATTTCGGCCTATTGGTTAAAAAATGAGCTGA
TTTAACAAAAATTTAACGCGAATTTTAACAAAATATTAACGTTTACAATTTC
AGGTGGCACTTTTCGGGGAAATGTGCGCGGAACCCCTATTTGTTTATTTTTC
TAAATACATTCAAATATGTATCCGCTCATGAATTAATTCTTAGAAAAACTCA
TCGAGCATCAAATGAAACTGCAATTTATTCATATCAGGATTATCAATACCAT
ATTTTTGAAAAAGCCGTTTCTGTAATGAAGGAGAAAACTCACCGAGGCAGT
TCCATAGGATGGCAAGATCCTGGTATCGGTCTGCGATTCCGACTCGTCCAAC
ATCAATACAACCTATTAATTTCCCCTCGTCAAAAATAAGGTTATCAAGTGAG
AAATCACCATGAGTGACGACTGAATCCGGTGAGAATGGCAAAAGTTTATGC
ATTTCTTTCCAGACTTGTTCAACAGGCCAGCCATTACGCTCGTCATCAAAAT
CACTCGCATCAACCAAACCGTTATTCATTCGTGATTGCGCCTGAGCGAGACG
AAATACGCGATCGCTGTTAAAAGGACAATTACAAACAGGAATCGAATGCAA
CCGGCGCAGGAACACTGCCAGCGCATCAACAATATTTTCACCTGAATCAGG
ATATTCTTCTAATACCTGGAATGCTGTTTTCCCGGGGATCGCAGTGGTGAGT
AACCATGCATCATCAGGAGTACGGATAAAATGCTTGATGGTCGGAAGAGGC
ATAAATTCCGTCAGCCAGTTTAGTCTGACCATCTCATCTGTAACATCATTGG
CAACGCTACCTTTGCCATGTTTCAGAAACAACTCTGGCGCATCGGGCTTCCC
ATACAATCGATAGATTGTCGCACCTGATTGCCCGACATTATCGCGAGCCCAT
TTATACCCATATAAATCAGCATCCATGTTGGAATTTAATCGCGGCCTAGAGC
AAGACGTTTCCCGTTGAATATGGCTCATAACACCCCTTGTATTACTGTTTAT
GTAAGCAGACAGTTTTATTGTTCATGACCAAAATCCCTTAACGTGAGTTTTC
GTTCCACTGAGCGTCAGACCCCGTAGAAAAGATCAAAGGATCTTCTTGAGA
TCCTTTTTTTCTGCGCGTAATCTGCTGCTTGCAAACAAAAAAACCACCGCTA
CCAGCGGTGGTTTGTTTGCCGGATCAAGAGCTACCAACTCTTTTTCCGAAGG
TAACTGGCTTCAGCAGAGCGCAGATACCAAATACTGTCCTTCTAGTGTAGCC
GTAGTTAGGCCACCACTTCAAGAACTCTGTAGCACCGCCTACATACCTCGCT
CTGCTAATCCTGTTACCAGTGGCTGCTGCCAGTGGCGATAAGTCGTGTCTTA
CCGGGTTGGACTCAAGACGATAGTTACCGGATAAGGCGCAGCGGTCGGGCT
GAACGGGGGGTTCGTGCACACAGCCCAGCTTGGAGCGAACGACCTACACCG
AACTGAGATACCTACAGCGTGAGCTATGAGAAAGCGCCACGCTTCCCGAAG
GGAGAAAGGCGGACAGGTATCCGGTAAGCGGCAGGGTCGGAACAGGAGAG
CGCACGAGGGAGCTTCCAGGGGGAAACGCCTGGTATCTTTATAGTCCTGTC
GGGTTTCGCCACCTCTGACTTGAGCGTCGATTTTTGTGATGCTCGTCAGGGG
GGCGGAGCCTATGGAAAAACGCCAGCAACGCGGCCTTTTTACGGTTCCTGG
CCTTTTGCTGGCCTTTTGCTCACATGTTCTTTCCTGCGTTATCCCCTGATTCTG
TGGATAACCGTATTACCGCCTTTGAGTGAGCTGATACCGCTCGCCGCAGCCG
AACGACCGAGCGCAGCGAGTCAGTGAGCGAGGAAGCGGAAGAGCGCCTGA
TGCGGTATTTTCTCCTTACGCATCTGTGCGGTATTTCACACCGCATATATGGT
GCACTCTCAGTACAATCTGCTCTGATGCCGCATAGTTAAGCCAGTATACACT
CCGCTATCGCTACGTGACTGGGTCATGGCTGCGCCCCGACACCCGCCAACA
CCCGCTGACGCGCCCTGACGGGCTTGTCTGCTCCCGGCATCCGCTTACAGAC
AAGCTGTGACCGTCTCCGGGAGCTGCATGTGTCAGAGGTTTTCACCGTCATC
ACCGAAACGCGCGAGGCAGCTGCGGTAAAGCTCATCAGCGTGGTCGTGAAG
CGATTCACAGATGTCTGCCTGTTCATCCGCGTCCAGCTCGTTGAGTTTCTCC
AGAAGCGTTAATGTCTGGCTTCTGATAAAGCGGGCCATGTTAAGGGCGGTT
TTTTCCTGTTTGGTCACTGATGCCTCCGTGTAAGGGGGATTTCTGTTCATGGG
GGTAATGATACCGATGAAACGAGAGAGGATGCTCACGATACGGGTTACTGA
TGATGAACATGCCCGGTTACTGGAACGTTGTGAGGGTAAACAACTGGCGGT
ATGGATGCGGCGGGACCAGAGAAAAATCACTCAGGGTCAATGCCAGCGCTT
CGTTAATACAGATGTAGGTGTTCCACAGGGTAGCCAGCAGCATCCTGCGAT
GCAGATCCGGAACATAATGGTGCAGGGCGCTGACTTCCGCGTTTCCAGACT
TTACGAAACACGGAAACCGAAGACCATTCATGTTGTTGCTCAGGTCGCAGA
CGTTTTGCAGCAGCAGTCGCTTCACGTTCGCTCGCGTATCGGTGATTCATTC
TGCTAACCAGTAAGGCAACCCCGCCAGCCTAGCCGGGTCCTCAACGACAGG
AGCACGATCATGCGCACCCGTGGGGCCGCCATGCCGGCGATAATGGCCTGC
TTCTCGCCGAAACGTTTGGTGGCGGGACCAGTGACGAAGGCTTGAGCGAGG
GCGTGCAAGATTCCGAATACCGCAAGCGACAGGCCGATCATCGTCGCGCTC
CAGCGAAAGCGGTCCTCGCCGAAAATGACCCAGAGCGCTGCCGGCACCTGT
CCTACGAGTTGCATGATAAAGAAGACAGTCATAAGTGCGGCGACGATAGTC
ATGCCCCGCGCCCACCGGAAGGAGCTGACTGGGTTGAAGGCTCTCAAGGGC
ATCGGTCGAGATCCCGGTGCCTAATGAGTGAGCTAACTTACATTAATTGCGT
TGCGCTCACTGCCCGCTTTCCAGTCGGGAAACCTGTCGTGCCAGCTGCATTA
ATGAATCGGCCAACGCGCGGGGAGAGGCGGTTTGCGTATTGGGCGCCAGGG
TGGTTTTTCTTTTCACCAGTGAGACGGGCAACAGCTGATTGCCCTTCACCGC
CTGGCCCTGAGAGAGTTGCAGCAAGCGGTCCACGCTGGTTTGCCCCAGCAG
GCGAAAATCCTGTTTGATGGTGGTTAACGGCGGGATATAACATGAGCTGTC
TTCGGTATCGTCGTATCCCACTACCGAGATGTCCGCACCAACGCGCAGCCCG
GACTCGGTAATGGCGCGCATTGCGCCCAGCGCCATCTGATCGTTGGCAACC
AGCATCGCAGTGGGAACGATGCCCTCATTCAGCATTTGCATGGTTTGTTGAA
AACCGGACATGGCACTCCAGTCGCCTTCCCGTTCCGCTATCGGCTGAATTTG
ATTGCGAGTGAGATATTTATGCCAGCCAGCCAGACGCAGACGCGCCGAGAC
AGAACTTAATGGGCCCGCTAACAGCGCGATTTGCTGGTGACCCAATGCGAC
CAGATGCTCCACGCCCAGTCGCGTACCGTCTTCATGGGAGAAAATAATACT
GTTGATGGGTGTCTGGTCAGAGACATCAAGAAATAACGCCGGAACATTAGT
GCAGGCAGCTTCCACAGCAATGGCATCCTGGTCATCCAGCGGATAGTTAAT
GATCAGCCCACTGACGCGTTGCGCGAGAAGATTGTGCACCGCCGCTTTACA
GGCTTCGACGCCGCTTCGTTCTACCATCGACACCACCACGCTGGCACCCAGT
TGATCGGCGCGAGATTTAATCGCCGCGACAATTTGCGACGGCGCGTGCAGG
GCCAGACTGGAGGTGGCAACGCCAATCAGCAACGACTGTTTGCCCGCCAGT
TGTTGTGCCACGCGGTTGGGAATGTAATTCAGCTCCGCCATCGCCGCTTCCA
CTTTTTCCCGCGTTTTCGCAGAAACGTGGCTGGCCTGGTTCACCACGCGGGA
AACGGTCTGATAAGAGACACCGGCATACTCTGCGACATCGTATAACGTTAC
TGGTTTCACATTCACCACCCTGAATTGACTCTCTTCCGGGCGCTATCATGCC
ATACCGCGAAAGGTTTTGCGCCATTCGATGGTGTCCGGGATCTCGACGCTCT
CCCTTATGCGACTCCTGCATTAGGAAGCAGCCCAGTAGTAGGTTGAGGCCG
TTGAGCACCGCCGCCGCAAGGAATGGTGCATGCAAGGAGATGGCGCCCAAC
AGTCCCCCGGCCACGGGGCCTGCCACCATACCCACGCCGAAACAAGCGCTC
ATGAGCCCGAAGTGGCGAGCCCGATCTTCCCCATCGGTGATGTCGGCGATA
TAGGCGCCAGCAACCGCACCTGTGGCGCCGGTGATGCCGGCCACGATGCGT
CCGGCGTAGAGGATCGAGATCGATCTCGATCCCGCGAAATTAATACGACTC
ACTATAGGGGAATTGTGAGCGGATAACAATTCCCCTCTAGAAATAATTTTGT
TTAACTTTAAGAAGGAGATATACATATGAGGGGGATGCTGCCCCTCTTTGA
GCCCAAGGGCCGGGTCCTCCTGGTGGACGGCCACCACCTGGCCTACCGCAC
CTTCCACGCCCTGAAGGGCCTCACCACCAGCCGGGGGGAGCCGGTGCAGGC
GGTCTACGGCTTCGCCAAGAGCCTCCTCAAGGCCCTCAAGGAGGACGGGGA
CGCGGTGATCGTGGTCTTTGACGCCAAGGCCCCCTCCTTCCGCCACGAGGCC
TACGGGGGGTACAAGGCGGGCCGGGCCCCCACGCCGGAGGACTTTCCCCGG
CAACTCGCCCTCATCAAGGAGCTGGTGGACCTCCTGGGGCTGGCGCGCCTC
GAGGTCCCGGGCTACGAGGCGGACGACGTCCTGGCCAGCCTGGCCAAGAAG
GCGGAAAAGGAGGGCTACGAGGTCCGCATCCTCACCGCCGACAAAGACCTT
TACCAGCTCCTTTCCGACCGCATCCACGCCCTCCACCCCGAGGGGTACCTCA
TCACCCCGGCCTGGCTTTGGGAAAAGTACGGCCTGAGGCCCGACCAGTGGG
CCGACTACCGGGCCCTGACCGGGGACGAGTCCGACAACCTTCCCGGGGTCA
AGGGCATCGGGGAGAAGACGGCGAGGAAGCTTCTGGAGGAGTGGGGGAGC
CTGGAAGCCCTCCTCAAGAACCTGGACCGGCTGAAGCCCGCCATCCGGGAG
AAGATCCTGGCCCACATGGACGATCTGAAGCTCTCCTGGGACCTGGCCAAG
GTGCGCACCGACCTGCCCCTGGAGGTGGACTTCGCCAAAAGGCGGGAGCCC
GACCGGGAGAGGCTTAGGGCCTTTCTGGAGAGGCTTGAGTTTGGCAGCCTC
CTCCACGAGTTCGGCCTTCTGGAAAGCCCCAAGGCCCTGGAGGAGGCCCCC
TGGCCCCCGCCGGAAGGGGCCTTCGTGGGCTTTGTGCTTTCCCGCAAGGAGC
CCATGTGGGCCGATCTTCTGGCCCTGGCCGCCGCCAGGGGGGGCCGGGTCC
ACCGGGCCCCCGAGCCTTATAAAGCCCTCAGGGACCTGAAGGAGGCGCGGG
GGCTTCTCGCCAAAGACCTGAGCGTTCTGGCCCTGAGGGAAGGCCTTGGCC
TCCCGCCCGGCGACGACCCCATGCTCCTCGCCTACCTCCTGGACCCTTCCAA
CACCACCCCCGAGGGGGTGGCCCGGCGCTACGGCGGGGAGTGGACGGAGG
AGGCGGGGGAGCGGGCCGCCCTTTCCGAGAGGCTCTTCGCCAACCTGTGGG
GGAGGCTTGAGGGGGAGGAGAGGCTCCTTTGGCTTTACCGGGAGGTGGAGA
GGCCCCTTTCCGCTGTCCTGGCCCACATGGAGGCCACGGGGGTGCGCCTGG
ACGTGGCCTATCTCAGGGCCTTGTCCCTGGAGGTGGCCGAGGAGATCGCCC
GCCTCGAGGCCGAGGTCTTCCGCCTGGCCGGCCACCCCTTCAACCTCAACTC
CCGGGACCAGCTGGAAAGGGTCCTCTTTGACGAGCTAGGGCTTCCCGCCAT
CGGCAAGACGGAGAAGACCGGCAAGCGCTCCACCAGCGCCGCCGTCCTGG
AGGCCCTCCGCGAGGCCCACCCCATCGTGGAGAAGATCCTGCAGTACCGGG
AGCTCACCAAGCTGAAGAGCACCTACATTGACCCCTTGCCGGACCTCATCC
ACCCCAGGACGGGCCGCCTCCACACCCGCTTCAACCAGACGGCCACGGCCA
CGGGCAGGCTAAGTAGCTCCGATCCCAACCTCCAGAACATCCCCGTCCGCA
CCCCGCTTGGGCAGAGGATCCGCCGGGCCTTCATCGCCGAGGAGGGGTGGC
TATTGGTGGCCCTGGACTATAGCCAGATAGAGCTCAGGGTGCTGGCCCACC
TCTCCGGCGACGAGAACCTGATCCGGGTCTTCCAGGAGGGGGGGGACATCC
ACACGGAGACCGCCAGCTGGATGTTCGGCGTCCCCCGGGAGGCCGTGGACC
CCCTGATGCGCCGGGCGGCCAAGACCATCAACTTCGGGGTCCTCTACGGCA
TGTCGGCCCACCGCCTCTCCCAGGAGCTAGCCATCCCTTACGAGGAGGCCC
AGGCCTTCATTGAGCGCTACTTTCAGAGCTTCCCCAAGGTGCGGGCCTGGAT
TGAGAAGACCCTGGAGGAGGGCAGGAGGCGGGGGTACGTGGAGACCCTCT
TCGGCCGCCGCCGCTACGTGCCAGACCTAGAGGCCCGGGTGAAGAGCGTGC
GGGAGGCGGCCGAGCGCATGGCCTTCAACATGCCCGTCCAGGGCACCGCCG
CCGACCTCATGAAGCTGGCTATGGTGAAGCTCTTCCCCAGGCTGGAGGAAA
TGGGGGCCAGGATGCTCCTTCAGGTCCACGACGAGCTGGTCCTCGAGGCCC
CAAAAGAGAGGGCGGAGGCCGTGGCCCGGCTGGCCAAGGAGGTCATGGAG
GGGGTGTATCCCCTGGCCGTGCCCCTGGAGGTGGAGGTGGGGATAGGGGAG
GACTGGCTCTCCGCCAAGGAGGGCAGCGGTGGCGTTGATAAACCCCAAAAG
CAAGAATTTGTTGGGATTTGCATAGTTAAATATCCAAAAAAACAAACCCAA
AAAGGCACAATAGTATCGAAAGCAATTTTAACTAGCTTAGATAGGGAATTG
CCTGTAGTATATTTCAACGATTTTGATTGGGAAATAGGCCATATATATAAAG
TATATGGAAAGCTTAAGAAAAACATAAAAACTGGTAAAATAGAATTTTTCG
CTGACAAAGTTGAGGAAGCAACATTAAAAGATCTAAAAGCTTTTAAAGGAG
AGGCCGATCACCACCACCACCACCACTAAGGATCCGAATTCGAGCTCCGTC
GACAAGCTTGCGGCCGCACTCGAGCACCACCACCACCACCACTGAGATCCG
GCTGCTAACAAAGCCCGAAAGGAAGCTGAGTTGGCTGCTGCCACCGCTGAG
CAATAACTAGCATAACCCCTTGGGGCCTCTAAACGGGTCTTGAGGGGTTTTT
TGCTGAAAGGAGGAACTATATCCGGAT
SEQ. ID. 12
TaqPolNeqSSBFull_F1
5′AACTTTAAGAAGGAGATATACATATGAGGGGGATGCTGCCCCTCTTTG3′
TaqPolNeqSSBFull_R1
5′TCATCAACGCCACCGCTGCCCTCCTTGGCGGAGAGCCAGTC3′
TaqPolNeqSSBFull_F2
5′AGGGCAGCGGTGGCGTTGATGATGAAGAGGAACTAATACAACTAATAATAG3′
TaqPolNeqSSBFull_R2
5′GCAAGCTTGTCGACGGAGCTCGAATTCGGATCCTTAGTGGTGGTGGTGGTGGTGATCGGCCT
CTCCTTTAAAAGCTTTTA3′
TaqPolNeqSSBII + III_F1
5′AACTTTAAGAAGGAGATATACATATGAGGGGGATGCTGCCCCTCTTTG3′
TaqPolNeqSSBII + III _R1
5′TCATCAACGCCACCGCTGCCCTCCTTGGCGGAGAGCCAGTC3′
TaqPolNeqSSBII + III F2
5′AGGGCAGCGGTGGCGTTGATGATGAAGAGGAACTAATACAACTAATAATAG3′
TaqPolNeqSSBII + III_R2
5′GCAAGCTTGTCGACGGAGCTCGAATTCGGATCCTTAGTGGTGGTGGTGGTGGTGATCGGCCT
CTCCTTTAAAAGCTTTTA3′
TaqPolNeqSSBIII_F1
5′AACTTTAAGAAGGAGATATACATATGAGGGGGATGCTGCCCCTCTTTG3′
TaqPolNeqSSBIII_R1
5′TTATCAACGCCACCGCTGCCCTCCTTGGCGGAGAGCCAGTC3′
TaqPolNeqSSBIII_F2
5′AGGGCAGCGGTGGCGTTGATAAACCCCAAAAGCAAGAATTTGTTG3′
TaqPolNeqSSBIII_R2
5′GCAAGCTTGTCGACGGAGCTCGAATTCGGATCCTTAGTGGTGGTGGTGGTGGTGATCGGCCT
CTCCTTTA3′

1. A TaqPol-NeqSSB polymerase comprising SEQ. ID 1-3.

2. A method for cloning TaqPol-NeqSSB polymerase comprising SEQ.ID 1-3, wherein insert DNA for cloning is obtained, which involves two independent PCR reactions:
the first amplification reaction yields a product with a nucleotide sequence corresponding to the gene sequence encoding the Taq DNA polymerase with an additional linker sequence and complementary to the 11 starting nucleotides of the NeqSSB protein at the C-end,
the second product contains the nucleotide sequence of the gene encoding the DNA-binding protein NeqSSB with additional nucleotides specific for the linker and 11 additional nucleotides complementary to the final nucleotide sequence of Taq polymerase at the N-end,
the isolated genomic DNA provides the template for the PCR reaction,
the products obtained in the two above-mentioned reactions are separated in agarose gel with ethidium bromide, and isolated from the gel.

3. The method of claim 2, wherein the products of two PCR reactions serve as inserts in a Gibson reaction, wherein:
pET30EKLIC plasmid is digested
to linearise the pET30EKLIC plasmid, it is digested with BamHI and NdeI (NEB) enzymes, which cut at two sites leaving the DNA ends non-complementary to each other,
the vector DNA digestion reaction is carried out for 2 h at 37° C. with addition of appropriate buffer,
the digested plasmid is separated electrophoretically and isolated,
the gene assembly reaction,
the Gibson reaction is carried out in a thermocycler at 50° C. for 60 minutes, where the mixture contains buffer, nucleotides, enzymes, sterile water, Insert I, Insert II, vector,
after the reaction, the mixture is added to freshly prepared E. coli TOP10 competent cells,
the resulting mixture is incubated on ice for 40 min, after this incubation time a heat shock is performed by placing the cell mixture for 60 s in a 42° C. thermoblock, followed by 2 min of incubation on ice, after the heat shock, the cells are incubated for 60 min at 37° C. with 600 ml LB, after that time the cells are centrifuged (10 min, 1800 rpm), 500 ml of the filtrate was discarded, the pellet was resuspended in the remaining supernatant and seeded onto LA plates supplemented with kanamycin, the plates were incubated for approximately 16 h at 37° C.

4. The method of claim 3, wherein to obtain a Taq-NeqSSB fusion protein, E. coli BL RIL cells are transformed using recombinant plasmid DNA pET30-TaqPol-NeqSSB, and production of the desired fusion protein is carried out, cultures with the addition of kanamycin and chloramphenicol are grown for 16 h at 37° C., rejuvenated and when the cultures reach OD600=0.5, IPTG is added to a final concentration of 0.1 mM; after induction, the cultures are grown for another 5 h, after which they are centrifuged (10 min, 5000 rpm) and subjected to purification by metalloaffinity; the results of protein production are analysed by polyacrylamide electrophoresis of protein under denaturing conditions.

5. An isolated recombinant plasmid comprising a fragment of the nucleotide sequence of the protein encoding the TaqPol-NeqSSBFull/II+III/III polymerase from 5076 to 8336 from the pET30EKLIC plasmid with SEQ. ID. 9, 5076 to 8159 from plasmid pET30EKLIC with SEQ. ID. 10, and from 5076 to 7886 from plasmid pET30EKLIC with SEQ. ID. 11.

6. An isolated pET30-TaqPol-NeqSSBFull/II+III/III plasmid comprising sequence SEQ.ID.9-11.

7. TaqPol-NeqSSBFull/II+III/III polymerase cloning primers comprising sequences SEQ.ID. 12-23.

8. A TaqPol-NeqSSBFull/II+III/III polymerase comprising SEQ. ID 1-3 for application to the replication of specific SARS CoV-2 virus sequences.
